(19) United States
(12) Patent Application Publication
(10) Pub. No.: US 2023/0375541 A1
(43) Pub. Date: Nov. 23, 2023

(54) PLASMONIC SENSORS AND ACTUATORS FOR IMAGING BIOLOGICAL MICROPARTICLES AND NANOPARTICLES

(71) Applicants: University of Houston System, Houston, TX (US); Board of Regents, The University of Texas System, Austin, TX (US)

(72) Inventors: Wei-Chuan Shih, Houston, TX (US); Nareg Ohannesian, Houston, TX (US); Steven H. Lin, Pearland, TX (US)

(73) Assignees: University of Houston System, Houston, TX (US); Board of Regents, The University of Texas System, Austin, TX (US)

(21) Appl. No.: 18/029,698

(22) PCT Filed: Oct. 4, 2021
(86) PCT No.: PCT/US2021/053317

§ 371 Date: Mar. 31, 2023

Related U.S. Application Data
(60) Provisional application No. 63/086,713, filed on Oct. 2, 2020.

(51) Int. Cl. G01N 33/543; B82Y 5/00; G01N 33/553; G01N 33/569; G01N 33/574

(52) U.S. Cl. CPC G01N 33/54346; B82Y 5/00; G01N 33/553; G01N 33/569; G01N 33/574

(57) ABSTRACT

Ultra near-field index modulated plasmonic nano-aperture label-free imaging methods and techniques are useful for imaging and detection of biological microparticles and nanoparticles such as circulating tumor exosomes (CTEs), bacteria and vimses. The methods and techniques utilize a high-density array of gold, silver, or gold/silver alloy nanodisks, in some cases on an undercut or invisible substrate. Given the relatively large nanodisk dimensions, the nanodisk array may feature a significantly blue-shifted LSPR extinction peak due to both far-field plasmonic coupling and substrate undercut. The ultra near-field imaging methods have the ability to image nanoparticles as small as 25 nm.

This application claims priority to U.S. Provisional Patent Application Ser. No. 63/086,713 entitled “Plasmonic Sensors and Actuators for Imaging Biological Microparticles and Nanoparticles,” filed Oct. 2, 2020, the entire contents of which are hereby incorporated by reference.

### BACKGROUND

This invention was made with government support under grant CBET-1605683 awarded by the National Science Foundation. The government has certain rights in the invention.

This disclosure pertains to imaging of biological microparticles and nanoparticles and particularly to the imaging and detection of circulating tumor exosomes (CTEs).

Circulating tumor exosomes (CTE) are nano-sized extracellular vesicles excreted by mammalian cells that circulate freely in the bloodstream of living organisms. Exosomes have a lipid bilayer that encloses genetic material used in intracellular communication (e.g., double-stranded DNA, micro-RNAs, and messenger RNA). Recent evidence suggests that dysregulation of this genetic content within exosomes has a major role in tumor progression and in the surrounding microenvironment. Although several genetic biomarkers have been validated for their diagnostic value, proteomic biomarkers can also provide a diagnostic value that is still being actively pursued. Recently, CTE have attracted intense attention due to its potential in complementing or even outperforming the outcomes of other circulating biomarkers such as circulating tumor cell (CTC), circulating tumor DNA (CTDNA), and traditional circulating protein biomarkers, which also aim to survey the tumor information from the blood. Detection of cancer biomarkers in the blood, known as “liquid biopsy”, can in principle increase accuracy by detecting the undetectable, or often known as “minimal residual”, disease (MRD). Although both CTC and CTDNA have been heavily pursued for cancer diagnostics and treatment monitoring, the utility of CTC has been limited to certain cancer types and has very low sensitivity. CTDNA is an emerging biomarker which has better sensitivity than CTC but is still limited for early diagnosis and MRD detection, and bears high cost and slow turnaround. The reason for their rarity is primarily owning to that both CTC and CTDNA derived from dead cancer cells, which are dwarfed by those derived from dead normal cells. Salient examples can be drawn from Co-I's recent CTDNA paper published in Nature Cancer on non-small cell lung cancer and Gastroenterology on esophageal cancer. Although the specific technology used is sensitive and specific, this approach is highly specialized, requires intricate informatics, and incurs great cost and long turnaround time, so barriers exist in its scalability and translational value. In addition, it is generally limited to cancer applications but not broadly to other diseases.

In contrast, exosomes exists at a much higher abundance than shed cells or DNA due to the fact that exosomes are actively excreted. In particular, cancer cells excrete exosomes into the blood circulation at a rate that is much higher than normal cells. Moreover, they play an important role in cell-to-cell communication by reprogramming neighboring cells that engulf and incorporate the molecular program encoded in the cargo genetic materials. Analyzing CTE content can yield direct insight into the state of the malignant parental cell. However, due to its small size (30-150 nm in diameter) and complexity (membrane and cargo), barriers exist for quantifying and characterizing the pool of CTE that reflects the disease state.

These barriers include complex protocols, as CTE isolation and purification require a set of tools and equipment completely different from that in the subsequent detection and quantitation. Thus, current workflow for CTE profiling involve a two-stage protocol, which increases labor and cost, reduces efficiency and sensitivity, and incurs delay. Label-free sizing/counting also lacks molecular specificity. Due to the small size, it is challenging to detect and count exosomes using traditional optical microscopy. Commercial and specialized nanoparticle tracking analysis (NTA) lacks molecular specificity and can only provide enumeration and size distribution. Fluorescence-based flow cytometry, on the other hand, does not have single unit sensitivity, and the results are averaged of many (˜100's-1000's) captured on microbeads. These factors lead to poor sensitivity and specificity due to high background signal from normal exosomes. Emerging technologies analyze up- and down-regulation of exosome surface antigen biomarkers, but their direct causal relationship to specific cancer types needs to be established. Using exosomal cargo D/RNA for diagnostics is much preferred, however, the sensitivity is too low due to bulk sampling with high background from normal exosomes. Additionally, similar to CTC, CTE cannot be directly amplified as CTDNA and analyzed by deep sequencing or polymerase chain reaction (PCR). Thus, to profile D/RNA in CTE cargo requires additional steps such as extraction and amplification, which again increases overall complexity, incurs high cost and long turnaround time, and leads to highly averaged results with low sensitivity.

Several approaches for extracting exosomes from bodily fluids have been developed including polymer/buffer-based precipitation, ultrafiltration, membrane affinity spin columns, ultracentrifugation, and immunological separation. However, none of these methods can produce pure CTEs. As a result, downstream sensitivity is poor because the large background from normal exosomes. A fundamental way to address this issue is to perform single exosome profiling.

To unlock the information in exosomes, methods that analyze the genetic or surface biomarkers are required. Enzyme linked immunosorbent array (ELISA) based Immunofluorescence, immunoblot imaging and immunofluorescence flow cytometry are common techniques used to analyze exosomes by targeting surface protein biomarkers. ELISA based Immunofluorescence captures and analyzes specific surface biomarkers by sandwiching exosomes between 2 complementary antibodies, one attached to an assay and the other to a fluorescent or catalyst label. A quantitative analysis is done through fluorescence or colorimetric response, but is a time-consuming process and does not reach sufficient detection limit. Another form of surface protein biomarker-based analysis is Chemiluminescence immunoblot imaging. This is similar to ELISA based immunofluorescence image but without an assay to immobilize the exosome on a specific region Immunofluorescence flow cytometry involves labeling exosomes with fluorescent markers so that light is first absorbed and then emitted in a different wavelength. A unique feature of this approach is exosome counting, where tens of thousands of exosomes can be quickly examined A common disadvantage of these methods that target the surface biomarkers is that they all have low diagnostic value. Surface protein that are linked to cancerous exosomes are not always unique. Majority of surface biomarkers are ubiquitous and can be found on normal exosomes, at a lower quantity but still cannot be separated fully.

The current state of technology for CTE analysis strives to identify and quantify specific exosomes of interest by the detection of the molecular cargo contents. Typically, next generation sequencing (NGS) or quantitative real time polymer chain reaction (qrt-PCR) are used to analyze dsDNA mutations or dysregulation in mRNA and miRNA expression compared to normal cell exosomes. This approach has insufficient sensitivity required for early detection and MRD analysis as mentioned previously. Another limitation to this approach is the non-selective process used for exosome extraction.

Several methods could be used to examining single exosomes, such as scanning electron microscopy, atomic force microscopy, or nanoparticle tracking assay (NTA). However, these techniques do not provide any molecular specificity and have minimal diagnostic value in clinical medicine.

Many emerging technological approaches have been implemented to provide better CTE analysis in the past decade, but the required sensitivity and specificity have not been reached for early detection and residual cancer detection where bulk sampling prevails. The best plasmonic sensor has a limit of detection in the hundreds of CTEs, which is far from single units. Many of the existing approaches first immobilized the CTE on a substrate which either has pre-defined bio-recognition elements or not. Existing molecular beacon based techniques either employs bulk sampling or lack single exosome resolution. They all rely on already isolated/purified exosome feeds without an integrated front-end function. Moreover, existing approaches either target surface proteomic biomarkers or internal genetic biomarkers, but not both.

Dielectric nanoparticles are considered transparent “phase” objects in optical physics, i.e., they contribute a slight variation to the local refractive index. Owing to their minute physical dimension, there are two fundamental issues preventing their facile characterization at single particle level by optical microscopy. First, because their scattering cross-section is extremely small, they can hardly stand out from the background, representing a detection challenge. Second, because of their size being much smaller than the optical diffraction limit, resolving individual units among a group of nanoparticles within close proximity is difficult, posing a resolution challenge. To overcome the detection challenge, dark-field illumination has been employed while suffering low light throughput thus slow imaging speed. To circumvent the resolution challenge, recent advances in superresolution imaging have gained considerable ground, but are limited to fluorescence microscopy. Non-fluorescent nanoparticles can flow through one by one in flow cytometry so there is no need to resolve. This type of serial measurement is usually in the dark-field mode with limited throughput. To increase throughput, intense light illumination has to be employed which could introduce adverse effects. Using a single pixel detector, they cannot take advantage of the rapid advances in camera technology for high-throughput, high-information content (HIC) imaging. In addition, any flow-through technology can only measure the nanoparticle as it travels by the detection area. This prevents time-lapse monitoring of the same nanoparticles. Besides, once a nanoparticle is measured, it is difficult to retrieve it for any additional measurement or processing. For label-free measurements, e.g., dark-field scattering, there is a lack of molecular information—only particle count and size distribution can be obtained. Label-free optical techniques also include Interferometric Scattering microscopy (iSCAT) and Interferometric Reflectance Imaging Sensor (IRIS). Although iSCAT has been shown to detect single molecules and gold particles as small as 2 nm, it does not provide size information and thus has been primarily employed for tracking applications. IRIS is based on traditional interference technique so the surface sensitivity is lower than plasmonic techniques. Nevertheless, both iSCAT and IRIS require an interferometry setup and a high quality laser. Labeled techniques require additional labeling and washing procedures of reporter labels such as fluorescent dyes, quantum dots, and gold nanoparticles, which increases complexity and cost. Fluorescence imaging, a commonly used labeling technique in biology, still faces challenges in imaging biological nanoparticles such as extracellular nanovesicles and viruses detection due to insufficient fluorescent photons and photobleaching.

Light excited surface plasmons (SP) have become the basis of many optical imaging and sensing techniques. Relying on propagating surface plasmon polariton (SPP), surface plasmon resonance (SPR) is a highly effective mechanism to detect minute refractive index (RI) changes on a gold thin. Because most detection targets have a RI higher than that of the ambient medium (e.g., air or water), SPR can detect surface binding without “labeling” with reporters such as fluorescent dyes, bypassing many potential labeling associated issues. Surface plasmon resonance imaging (SPRI) broadens the repertoire of SPR sensing by incorporating an imaging component. However, SPRI typically cannot achieve diffraction-limited lateral resolution with respect to the excitation wavelength due to the wave propagating nature of SPP that “smears” the point spread function. Recently, improved spatial resolution has been demonstrated by interferometric SPRI with image processing. The concept of digital holography has also been implemented to image single nanoparticles by SPR with near-field optics. Longitudinally, the evanescent field of SPP extends into the medium with a lie distance ˜240 nm, which can be considered as the first order approximation of its sensing range above the gold film. A predominant SPRI instrument configuration is based on the Kretschmann design where a prism is employed to provide total internal reflection (TIR) excitation, a required condition for momentum matching. It is worth noting that SPRI can only sense a few hundreds of nanometers above the gold surface, so it requires another imaging modality for the parts of specimen outside of this distance. For example, a standard SRPI system constructed based on an inverted microscope requires two light sources, one to provide the bottom-up TIR excitation, and the other a top-down transmission illumination. If a single camera is employed for detection, it must be time-shared by the two imaging modalities.

Localized surface plasmons (LSP), in contrast, refers to non-propagating SPs localized in metallic nanostructures and nanoparticles with a size comparable or smaller than the excitation wavelength. Similar to SPR, LSPR is also sensitive to the surface RI changes and has been heavily pursued in optical sensing of surface molecular binding. Due to the non-propagating nature of LSP, LSPR imaging (LSPRI) would address a key drawback in SPRI by eliminating the smearing effect, thus making diffraction-limited lateral resolution possible. Longitudinally, LSPRI also features shorter sensing distance from the surface, which can provide better sensitivity toward the surface compared to SPRI. Nevertheless, these tantalizing capabilities have not been fully realized. To date, LSPRI embodiments utilize colloidal nanoparticles or nanostructured substrates as sparse sensing units in the form of dark-field scattering microscopy. In the former case where colloidal nanoparticles are employed, their distribution is not pre-arranged nor controllable. In the latter case, arrays of sparsely distributed nanostructures such as pillars, posts, spikes, etc., have prevented continuous lateral (i.e., in the x-y plane) sampling. Compared to how SPRI is employed, where the gold substrate surface can be 100-percent utilized for continuous sampling, the existing LSPRI can only provide laterally sparse images with small imaging fill factor. Failure to supply continuous sampling has several drawbacks such as lower efficiency, missing spatial context, and “blind” to anything outside the sensing near-field both laterally and longitudinally.

### SUMMARY

The present disclosure relates generally to methods and systems for imaging of biological microparticles and nanoparticles, particularly to imaging and detection of micro and nano-vesicles such as circulating tumor exosomes (CTEs) or circulating non-tumor exosomes, or pathogens such as bacteria and viruses.

Exosomes are released by all types of mammalian cells, such as blood cell, endothelial cells, immunocytes, platelets and muscle. Exosome forms an intercellular communication channel and is responsible for the regulation of bioactivities of recipient cells through the transportation of lipids, proteins and nucleic acids while circulating in extracellular space. Several reports have been shown that exosome plays a major role in immune response, tumor progression and neurodegenerative disorders, cardiovascular diseases, stem cell research, drug delivery, maternal health, liver injury, and many others. Upon this discovery, a particular effort has been to identify CTE derived biomarkers, surface proteins (SP) and genetic biomarkers, towards early detection and post-treatment prognosis of cancer. Colon cancer has differentially expressed miRNA-125, 320-L and 193a; SP CD63, Alix, TSG101, CD81 and CD147 biomarkers. Bladder cancer has been reported for miRNA-146 and 375; SP Apo B biomarkers. Lung cancer has shown miRNA-126, 21, 155 and 16; SP CD9. Pancreatic cancer has miRNA 1246, 4644, 3976 and 4306; SP CD446, Tspan8, GPC1, EpCAM and CD104. Breast cancer has miR-1246, 21, 378e and 143; SP CD63, CD81, Hsp70, and Alix. Ovarian cancer cell line has miRNA 32b, 29a, 30d, 205 and 720 as high potential biomarkers. It also has SP CD81, CD24 Ca125, EpCAM, EGFR, MUC18 and CLDN3 biomarkers. Prostate cancer differentially expresses miRNA 1290 and 375; SP CD73 biomarkers. Gastric cancer has miRNA 451 and lncRNA UEGC1; SP CA19-9, CA72-4 and CA12-5 biomarkers. Liver cancer has miRNA 122-5p, 7a-5p, 199a-3p, 18a, 221, 222 and 224; SP EpCAM, CD144, CD63, CD9 and CD81 biomarkers. Furthermore, tumor specific mutations in dsDNA and differentially expressed mRNA are also packaged in CTEs that could also be specifically detected.

The diagnostic value of these various biomarkers varies quite significantly. Although surface protein biomarkers appear less abundant on healthy cell derived exosomes compared to the cancerous ones, they alone cannot be the sole biomarkers used towards cancer diagnosis. However, antigen-specific antibodies can be used to isolate exosomes with high efficiency. Coupled with the analysis of the genetic biomarkers packaged within the exosome would provide robust clinical diagnostic value. A new technology that can detect both surface protein and genetic biomarkers would provide unprecedented capabilities in early and residual cancer detection.

In particular, this disclosure relates to ultra near-field index modulated plasmonic nano-aperture label-free imaging methods and techniques that address existing issues for present SPRI and LSPRI techniques and are useful for imaging and detection of biological microparticles and nanoparticles such as CTEs and viruses. On one hand, these ultra near-field imaging methods can produce diffraction-limited lateral resolution free of the previously mentioned smearing effect in SPRI. These methods also have higher surface sensitivity due to the LSPR decay length being shorter than that of SPR. In some embodiments its system configuration is identical to a standard bright-field microscope using a trans-illumination tungsten-halogen lamp. Therefore, the present methods and techniques simultaneously image everything within the microscope objective's depth of focus with a single lamp source and a single camera. However, the intensity ratio (IR) increases as the target becomes closer to the imaging substrate. Furthermore, the methods and techniques described herein address the sparse sampling issue in dark-field LSPRI and provide dense sampling with a large imaging fill factor. In other words, these techniques can provide a panoramic view both laterally and longitudinally—overcoming the lack of imaging depth for both SPRI and LSPRI, and the insufficient lateral sampling for LSPRI.

In preferred embodiments, the present methods and techniques have been demonstrated on a high-density arrayed nanodisks on an “invisible” substrate. The arrayed nanodisks may be made of gold, a gold/silver alloy, or silver. In preferred embodiments the nanodisks may be shaped as circles, ovals, squares, triangles, rods, diamonds, or ellipses. In preferred embodiments, the nanodisks in the array measure between 100 and 1000 nm in diameter, preferably about 360 nm in diameter, and 20 to 150 nm in thickness, preferably about 50 nm in thickness. The edge-to-edge distance between nanodisks can vary depending on the disk diameter. In some embodiments the nanodisks in the array are positioned apart from each other by about 35 nm in edge-to-edge distance, for exemplary 130 nm diameter disks, or up to 100 nm in edge-to-edge distance between exemplary 360 nm disks. The high-density nanodisk array may be fabricated using nanosphere lithography followed by a self-aligned substrate undercut. In particular, a large portion of the glass substrate under the gold nanodisks is removed, which results in the nanodisks sitting on “posts” where the posts have a diameter of about 200 nm and a height of about 150 nm.

In preferred embodiments utilizing arrayed gold nanodisks, the transformation of arrayed gold nanodisks (AGN) to arrayed gold nanodisks on invsibisble substrates (AGNIS) utilizes an undercut process that has been studied using a 460 nm pitch array. Various degrees of undercut are obtained by stopping the etching process at selected times, which produces a series of varying extinction spectra and corresponding varying SEM images. In preferred examples of the undercut process, the radial and vertical etch rate was calculated to be 1.28 nm/s and 2.11 nm/s, respectively based on SEM images. During successive undercuts, the LSPR peak blue-shifted from 820 nm to a plateau value of 688 nm when the radial undercut distance reached 100 nm. In this example, since the nanodisk diameter was 350 nm, the nanodisk after the undercut process sat on underlying glass posts with a top diameter of 190 nm, which provided sufficient adhesion. Further undercut did not result in additional blue-shifts, suggesting the substrate effect was completely removed with a radial undercut distance of 100 nm. The greatest amount of radial undercut that was accomplished was ˜130 nm with glass posts having a top diameter of 90 nm, beyond which the nanodisks failed to adhere to the glass posts. Even after undercutting, the far-field, radiative coupling is still present for AGNIS although the substrate effect has been eliminated. For the AGN before undercut, a high energy mode at ˜551 nm was previously identified as a split mode due to the asymmetric superstrate/substrate configuration. This peak gradually diminished during the undercut process and disappeared eventually at 100 nm radial undercut, providing an independent proof that the substrate effect had been entirely removed.

In preferred embodiments the high-density nanodisk array features a significantly blue-shifted LSPR extinction peak of at least 688 nm in air due to both far-field plasmonic coupling and substrate undercut. The blue-shifted LSPR peak provides better diffraction-limited resolution and utilization of high quantum yield in silicon-based cameras. In alternative embodiments, substrate undercut in the nanodisk array might not be necessary when the nanodisks are made smaller. However, smaller nanodisks exhibit less radiative coupling, so the magnitude of blue-shift might not be sufficient. Therefore, undercut still represents an indispensable means and an additional “knob” to fine-tune and achieve the optimal blue-shifts. The current ultra near-field imaging methods have the ability to image dielectric nanoparticles as small as 25 nm using, in preferred embodiments, a standard transmission bright-field microscope with a tungsten-halogen lamp. In addition to ultrahigh sensitivity to deep sub-100 nanoparticles, the current techniques can also provide their size information. Furthermore, using the arrival time difference in a dynamic imaging mode, individual nanoparticles in a cluster with interparticle distance well below the diffraction limit of the current optical system (330 nm) can be counted. Moreover, the longitudinal distance between a nanoparticle and high-density nanodisk array can be monitored using the dynamic imaging mode.

In preferred embodiments, the high-density nanodisk array is utilized as part of an array of nanoplasmonic sensors in a microfluidic channel, where the high-density nanodisk array is functionalized to allow for capture and precise detection of biological micro and nanoparticles. To facilitate exosome detection and register an get an exact number of detected target particles, it is crucial to immobilize the biological micro or nanoparticle on the surface of the high-density nanodisk array. Targeting the surface protein with antibodies, it is possible to immobilize the biological micro or nanoparticle and also determine the expression of different surface proteins.

### BRIEF DESCRIPTION OF THE DRAWINGS

FIG. 1 shows (a) a schematic for a general micro/nanofabrication process for a nanoplasmonic sensor including arrayed gold/silver alloy nanodisks, (b) exemplary surface functionalization of the nanodisks and immuno-enrichment for capture of circulating tumor exosomes (CTEs), and (c) a schematic for an alternative general scheme for a micro/nanofabrication process for a nanoplasmonic sensor where the nanodisks are prepared with a substrate undercut.

FIG. 2 shows schematics of an exemplary needle device including nanoplasmonic sensors for detecting biological micro and nanoparticles in (a) sideview and (b) distal view facing the needle.

FIG. 3 shows scanning electron microscopy (SEM) images of nanoporous gold disks (NPGD) of various diameters: (a) 200, (b) 300, (c) 400, (d) 500 nm, and of (e) NPGD viewed from 45° view to show its 3D porous network.

FIG. 4 shows NPGD nanoarrays of various configurations: (a) large-scale NPGD array fabricated by NSL; (b) hexagonal nanoarray configuration by NSL; (c) squared nanoarray with 100 nm disk diameter and 100 nm edge-to-edge spacing; (d) squared nanoarray with 200 nm disk diameter and 100 nm edge-to-edge spacing.

FIG. 5 shows refractive index sensitivity of a NPGD nanoarray relative to peak shift.

FIG. 6 shows (a) SEM image of exemplary nanodisk array, (b) SEM image of exemplary nanodisk array with edge to edge distance of 500 nm showing undercut glass substrate, (c) LSPR extinction curve of bare nanodisk array in different media alongside the imaging wavelength range labeled with arrows, and (d) LSPR peak in water blue shifted from 830 nm to 690 nm after substrate undercut.

FIG. 7 shows a schematic of an optical setup for plamonic nano-aperture imaging.

FIG. 8(a)-(g) shows images of polystyrene (PS) beads prepared using preferred embodiments of the plasmonic nano-aperture imaging techniques described herein, where the PS beads were of the sizes (a) 750 nm, (b) 460 nm, (c) 300 nm, (d) 200 nm, (e) 100 nm, (f) 50 nm and (g) 25 nm.

FIG. 8(h)-(l) shows images of PS beads prepared with standard bright-field microscopy, where the PS beads were of the sizes (h) 750 nm, (i) 460 nm, (j) 300 nm, (k) 200 nm, and (1) 100 nm.

FIG. 9(a)-(g) shows IR histograms of images of polystyrene (PS) beads prepared using preferred embodiments of the plasmonic nano-aperture imaging techniques described herein, where the PS beads were of the sizes (a) 750 nm, (b) 460 nm, (c) 300 nm, (d) 200 nm, (e) 100 nm, (f) 50 nm and (g) 25 nm.

FIG. 9(h)-(k) shows IR histograms of images of PS beads prepared with standard bright-field microscopy, where the PS beads were of the sizes (h) 750 nm, (i) 460 nm, (j) 300 nm, and (k) 200 nm.

FIG. 9(l) shows a corresponding IR histogram of background from the squared regions in FIG. 7.

FIG. 10 shows IR versus nanoparticle diameter when using preferred embodiments of the plasmonic nano-aperture imaging techniques described herein (squares) and using bright-field microscopy (circles).

FIG. 11 shows detected particle pixel illumination grid for particle sizes (a)-(d) 25 nm, (e) 50 nm, (f) 100 nm, (g) 200 nm, (h)-(i) 300 nm, (j) 460 nm, and (k) 750 nm.

FIG. 12(a)-(f) shows IR when using preferred embodiments of the plasmonic nano-aperture imaging techniques described herein for PS beads of diameter (a) 25 nm, (b) 50 nm, (c) 100 nm, (d) 200 nm, (e) 300 nm and (f) 460 nm.

FIG. 12(g) shows IR for PS beads of diameter 750 nm when using preferred embodiments of the plasmonic nano-aperture imaging techniques described herein (circles) and when using bright-field imaging (squares).

FIG. 12(h) shows particle settlement time (r) vs. particle size where r was calculated from the points labeled 1 to 2 for all particle sizes in (a)-(g).

FIG. 12(i) shows FDTD simulation of IR vs. distance between a 50 nm particle and an exemplary high-density nanodisk array.

FIG. 13(a)-(b) shows images of a detected 25 nm PS bead at (a) selected time frame (i) and (b) selected time frame (ii).

FIG. 13(c)-(d) shows images of a detected 100 nm PS bead at (a) selected time frame (iii) and (b) selected time frame (iv).

FIG. 13(e)-(f) shows IR versus time showing settling of PS beads (e) of diameter 25 nm including time frames (i) and (ii) and (f) of diameter 100 nm including time frames (iii) and (iv).

FIG. 14 shows general structural schematics for a single well microfluidic arrayed nanoplasmonic sensor and actuator (MANSA).

FIG. 15 shows (a) LSPR extinction spectra and (b) LSPR peak shifts after successive steps of surface functionalization of an exemplary MANSA.

FIG. 16 shows (top) images of PS nanoparticles of diameter 100 nm and 25 nm and (bottom) intensity contrast for images of 100 nm, 50 nm, and 25 nm PS nanoparticles on an exemplary MANSA.

FIG. 17 shows (a) images of detected single exosomes using preferred embodiments of the imaging techniques and successive functionalized MANSA described herein and (b) intensity changes for successive functionalization steps of MANSA until exosome binding.

FIG. 18 shows intensity changes for binding of CTEs over time using preferred embodiments of the imaging techniques and successive functionalized MANSA described herein.

FIG. 19 shows averaged LSPR peak shifts relative to concentration of exosomes captured and imaged using preferred embodiments of the imaging techniques and successive functionalized MANSA described herein.

FIG. 20 shows nanoplasmonic enhanced fluorescence on NPGD relative to gold disks, gold film, and glass using the fluorophores (A) R6G, (B) Cy3, and (C) IRDye.

FIG. 21 shows fluorescence versus concentration of targeted miRNA using preferred embodiments of the imaging techniques and MANSA described herein functionalized with molecular beacon probes for binding the targeted miRNA.

FIG. 22 shows IR and corresponding LSPR peak shifts recorded after different steps of surface functionalization of an exemplary high-density nanodisk array.

FIG. 23(a)-(b) shows (a) size distribution of exosomes used for imaging analysis and (b) image of detected exosomes using preferred embodiments of plasmonic imaging techniques after settlement and washing.

FIG. 23(c)-(f) shows exosome detection in a marked location from FIG. 22(b) after (c) 20 min, (d) 40 min, (e) 80 min, and (f) 120 min of particle settlement time.

FIG. 23(g)-(h) shows (g) number of detected exosomes versus time after washing in marked 100 μm×100 μm area and (h) compiled histogram of detected exosome IR from FIG. 22(b).

FIG. 24(a)-(d) shows (a), (c) fluorescence image of exosomes on functionalized gold nanodisk array surface and (b), (d) image of exosomes on functionalized gold nanodisk array surface using preferred embodiments of plasmonic imaging techniques.

FIG. 25 shows profiles of exosome populations (cancerous H460 and non-cancerous 293A) defined by surface protein (CD63, CD9 and CD81) detected by monitoring the percentage of exosomes remaining on the surface of a functionalized gold nanodisk array after washing.

FIG. 26 shows (a) calibration curve of IR to size using polystyrene beads and (b) comparison of size distribution recorded via Nanosight particle tracking to size calculated using plasmonic imaging techniques.

### DETAILED DESCRIPTION OF PREFERRED EMBODIMENTS

The present disclosure relates generally to methods and systems for imaging of biological microparticles and nanoparticles, particularly to imaging and detection of micro and nano-vesicles such as circulating tumor exosomes (CTEs) or circulating non-tumor exosomes, or pathogens such as bacteria and viruses.

Preferred embodiments described herein relate to ultra near-field index modulated plasmonic nano-aperture label-free imaging methods and techniques that can produce diffraction-limited lateral resolution free of the smearing effect in SPR imaging. The imaging methods and techniques disclosed herein also have higher surface sensitivity due to the LSPR decay length being shorter than that of SPR. In preferred embodiments, the system configuration is identical to a standard bright-field microscope using a trans-illumination tungsten-halogen lamp instead of a laser or other high-intensity light sources. Therefore, the present methods allow for wide-field imaging over everything within the microscope objective's depth of focus with a single lamp source and a single camera. However, the intensity increases as the target becomes closer to the imaging substrate. Other narrow band light sources, such as LED, laser, SLD, and filtered plasma light can also be utilized in the present imaging methods. On a separate front, the present techniques address the sparse sampling issue in LSPR imaging by achieving dense sampling with a large imaging fill factor. The bright-field approach provides much higher light throughput compared to dark-field microscopy. Overall, this technique can provide a panoramic view both laterally and longitudinally—overcoming the lack of imaging depth for both SPR and LSPR imaging and the insufficient lateral sampling for LSPR imaging. The present methods and techniques can size single nanoparticle down to 25 nm, count individual nanoparticles in a cluster, and dynamically monitor single nanoparticles approaching the plasmonic surface down to the millisecond timescale.

Preferred embodiments described herein utilize a high-density nanodisk array, a polycrystalline array comprising nanodisks of gold, gold/silver alloy, or silver. In preferred embodiments the nanodisks may be shaped as circles, ovals, squares, triangles, rods, diamonds, or ellipses. In preferred embodiments the nanodisks may be nanoporous gold disks. In preferred embodiments, the nanodisks in the array measure between 100 and 1000 nm in diameter, preferably about 360 nm in diameter, and 20 to 150 nm in thickness, preferably about 50 nm in thickness. The edge-to-edge distance between nanodisks (i.e., the gap size) can vary depending on the disk diameter but the edge-to-edge distance should always be less than the disk diameter. In some embodiments the nanodisks in the array are positioned apart from each other by about 35 nm in edge-to-edge distance, for exemplary 130 nm diameter disks, or up to 100 nm in edge-to-edge distance between exemplary 360 nm disks. In some embodiments the substrate beneath the nanodisks in the array is partially removed, so each nanodisk is positioned on what is essentially a post of substrate. Undercut nanodisks have higher sensitivity, but non-undercut substrates will work at a longer wavelength. When the nanodisks are undercut, the substrate posts preferably have diameters of about 200 nm and heights of about 150 nm.

Preferred embodiments described herein also utilize an optical set-up that includes a tungsten-halogen lamp, a condenser, and an inverted microscope. Transmitted light passes through an infinity corrected water immersion lens with a 1.2 numerical aperture. Light exiting the side-port is relayed to a camera device such as an electron multiplied charge coupled device via a 4f system, with a bandpass filter with 650-670 nm passband at its Fourier plane. Other camera devices that may be used include CCD, CMOS, sCMOS, surveillance, and smartphone cameras.

Preferred embodiments of the plasmonic nano-aperture label free imaging techniques described herein utilize high-density plasmonic nanodisk arrays with a gap size that is less than the disk diameter. In preferred embodiments, the gap size is only about ⅓ to ½ of the disk diameter. Preferred embodiments also utilize bright field illumination, though the light source may vary. Preferred embodiments also require either the use of a filter to select a spectral band at near the half point of the LSPR peak or a narrow band light source centered at the same wavelength. The LSPR position is variable. The camera used can vary and can be, for example, CCD, EMCCD, CMOS, sCOMS, InGaAs, or the like. The imaging modality can also vary and can be, for example, phase contrast, dic, polarization, and the like.

The light scattering cross-section (ascatt) of a spherical particle is proportional to the 6th power of the particle diameter (d) given by σₛₑₐₜₜ=(2π⁵d⁶n_{med}⁴/3λ_{inc}⁴)*|(m²−1)/(m²+2)|, where n_{med} is the refractive index of the medium surrounding the particle, and m is the ratio of the refractive indices of the particle and medium. Unlike most light scattering techniques, the present technique does not solely rely on scattered light to detect nanoparticles. With the trans-illumination geometry, the camera receives transmitted light passing through the nanodisk array after a bandpass filter. The bandpass filter is located at near the half-max wavelength on the left shoulder of the nanodisk array's LSPR extinction curve. When an imaging target resides outside the nanodisk array's longitudinal sensing range, i.e., too far away from the surface, it will show up as a standard light scattering object with its intensity reduced when transmitted through the nanodisk array. However, as the imaging target approaches the nanodisk array, the elevated local RI causes the LSPR extinction curve to red shift. The LSPR red shift causes increased light transmission within the imaging wavelength range and acts as if a virtual nano-aperture is formed right beneath the target. The nano-aperture allows higher transmission for both the nanoparticle scattered light and the unscattered incidence light. It is important to recognize that the increased transmission of the unscattered incidence light may be crucial for detecting weakly scattering nanoparticles (sizes<200 nm). In alternative embodiments, a bandpass filter can be selected to be on the red (i.e., longer wavelength) side of the LSPR weak, whereby the LSPR red shift would effectively lower the transmission of nanoparticle scattered light and unscattered incidence light.

A broad range of microscopy imaging modalities can be used in connection with preferred embodiments of the imaging techniques described herein. These include, for example, phase contrast, digital interference contrast, diffraction phase microscopy, polarization microscopy, quantitative phase imaging, and interference scattering microscopy.

The plasmonic nano-aperture label free imaging techniques can be used as part of a technological platform for streamlined, scalable, and comprehensive analysis of biological micro or nanoparticles such as exosomes. In a first step, an aliquot of blood serum/plasma is first delivered to a microfluidic chip with multiple micro-wells. At the bottom of each micro-well sits microfluidic arrayed nanoplasmonic sensors and actuators (“MANSA”) including one or more nanodisk array chips with a cocktail of specific surface functionalization for selective enrichment of exosomes from any source. The size (3×3) of the micro-well matrix is scalable and individual MANSA can be surface functionalized independently for selective capture. Laser controlled nanoplasmonic micro- and nanobubble actuators will be employed on MANSA to generate directed flow to concentrate and isolate exosomes toward the bottom nanoplasmonic surface. Once immobilized by immuno-capturing, individual unit will cause the localized surface plasmon resonance (LSPR) peak of the underlying nanostructure to red-shift. Dynamic monitoring of the exosome binding/capturing on MANSA will be recorded by a wide-field narrow-band imaging system designed to acquire intensity jumps due to the LSPR red-shifts at diffraction-limited spatial resolution. This is a significant improvement over traditional SPR imaging where the optical point spread function is “smeared” due to the propagating SPR. The endpoint of this procedure produces a map of exosome distribution with both location registration and enumeration from a series of in situ binding image analysis using centroid locating akin to localization superresolution techniques such as PALM/STROM. To improve detection and quantitation reproducibility and robustness, a spectroscopic imaging system can be employed after all the exosomes bound to the MANSA. This system will acquire an extinction spectrum from each spot (500×500 nm²) over the entire active region, producing a 3-D dataset (x-y-A), which complement the dynamic imaging data acquired previously.

In a second step, liposome-encapsulated molecular beacons or other detection elements are delivered into the micro-wells where the biological micro or nanoparticles have been immobilized. The liposomes may be similar in size to individual exosomes, thus can provide a size-matched fusion and delivery of molecular beacons. In addition to the liposomal delivery, nanoplasmonic membrane permeability modulation and electroporation are used. The molecular beacon probes “turn on” upon hybridization with the target D/RNA sequence. Other types of detection elements besides molecular beacon probes may be used which also effectively “turn on” upon hybridization with the target D/RNA sequence, such as gold nanoparticles, surface-enhanced Raman scattering labels, and quantum dots. The fluorescence intensity will be further enhanced by the LSPR-induced electrical field concentration on MANSA, thus providing a signal intensity boost compared to standard beacons.

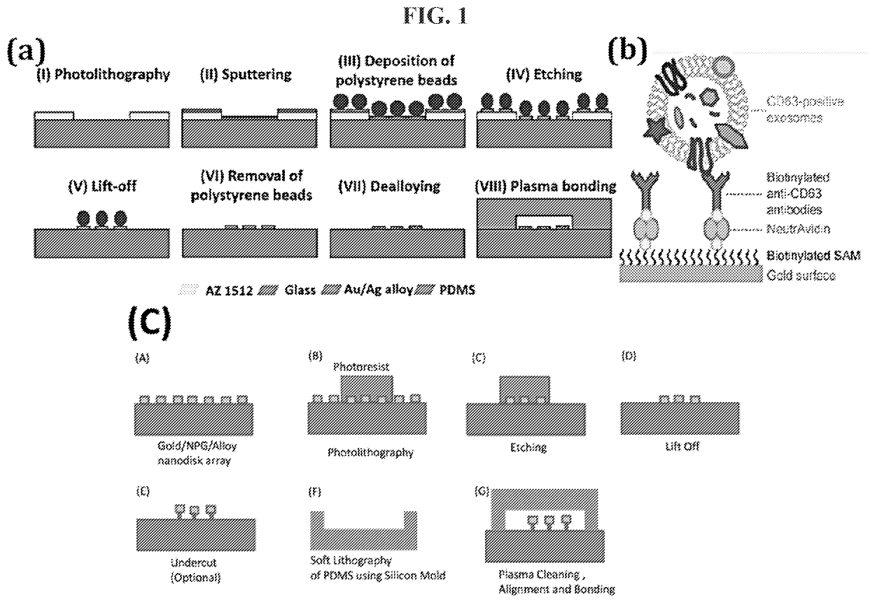

A highly reproducible micro/nanofabrication process flow is used to fabricate the microfluidic arrayed nanoplasmonic sensor & actuator (MANSA) platform consisting of closely-packed array of nanodisks. Arrayed nanodisks using multi-metal such as gold, nanoporous gold (NPG), silver and gold-silver alloy are fabricated with co-design of the multi-micro-well microfluidic device. The microfluidic chip consists of polydimethylsiloxane microchannels and chambers with multiple inlets and outlets for injection of various molecules and target solutions. FIG. 1(a) shows a general scheme for a micro/nanofabrication process for a microfluidic chip. Briefly, Step (I) patterns the regions where the MANSA will be situated. Step (II) deposits a metal or alloy layer of choice. Step (III-VI) patterns the metal layer into nanodisks of desirable diameter. As shown here, an effective nanosphere lithography technique is indicated. Electron-beam lithography is employed for different array configurations. Step (VII) dealloys the Au/Ag alloy by selectively leaching Ag using nitric acids to facilitate the formation of NPG. Step (VIII) bonds a PDMS microchannel/chamber construct that has been made using soft lithography to the ANS completed in Step (VII).

The nanodisk surfaces are functionalized with recognition elements that are known to be present on the circulating micro or nano-vesicles being detected, such as antibodies that are known to be up-regulated on exosomes of different cancer origins. The recognition elements comprise proteins such as antibodies or oligonucleotide probes such as aptamers. As shown in FIG. 1(b), in one example, the functionalization starts by forming a self-assembled monolayer of a mixture of biotin-PEG-SH and PEG-SH with a ratio of 3:1. Biotinated antibodies are then linked to the biotin-PEG-SH via neutravidin. As an alternative, EDC-NHS coupling can be used to immobilize the antibodies. The functionalization allows the nanodisk array to bind the biological microparticles or nanoparticles being detected, which may be micro and nano-vesicales such as circulating tumor or non-tumor exosomes, or which may be pathogens such as bacteria or viruses.

FIG. 1(c) shows an alternative general scheme for a micro/nanofabrication process for a microfluidic chip where the nanodisks are prepared with a substrate undercut.

To further implement the technique, a nanoplasmonic micro/nanobubble concentrator/isolator is utilized. The microbubble is generated by the conversion of absorbed light into heat via nanoplasmonics. The microbubble is capable of generating local flow circulation to bring the target biological particles down to the MANSA surface, while leaving larger vesicles or residual cells in the bulk flow. This approach will effectively deal with the transport limit in the micro-well and improve binding efficiency. Preferred size selection resolution allows it to concentrate only vesicles<200 nm.

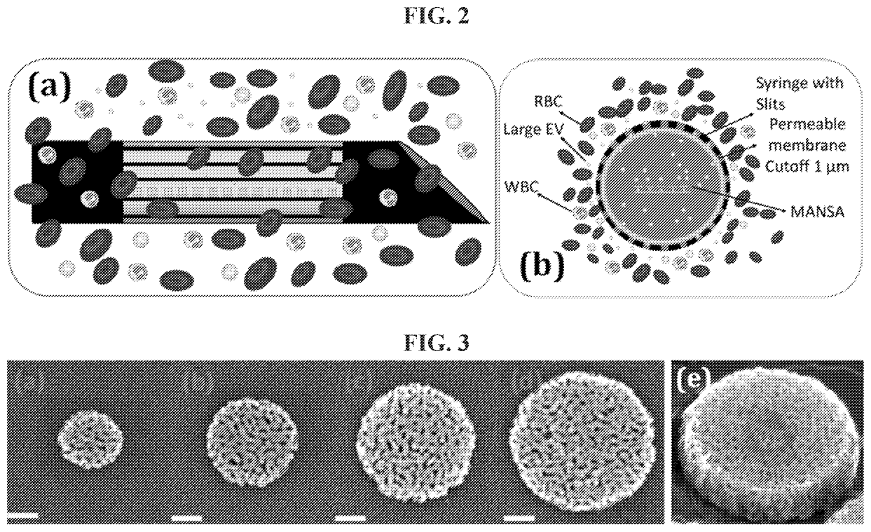

Additional implementations also utilize an integrated needle device for robust fluid sampling. Currently, all exosome profiling techniques require blood withdrawal. An integrated MANSA (iMANSA) needle device can be used for robust fluid sampling to eliminate blood withdrawal and processing, which reduces yield and damages the biologic material. A schematic for a needle device is shown in FIG. 2(a)-(b). A strip-shaped MANSA chip is placed inside a porous membrane inside a slotted needle. When the syringe is manually drawn, the fluid will enter the needle through the membrane which has a pore size of 1 μm. Cells and larger vesicles will not enter the needle. Once enough fluid enters the needle, the syringe can be pushed back to eject the fluid while opening up any clogged pores. A few pull-push cycles effectively enhance sampling of the circulating blood volume and improve capture of the biological micro or nanoparticles in the filtrate.

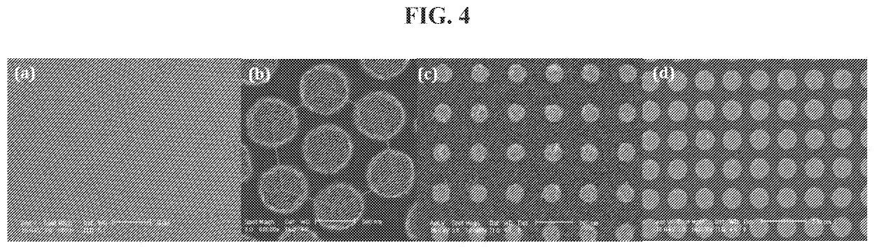

As shown in FIG. 3(a)-(d), a single NPG disk with diameter tunable from 200 to 500 nm, thickness 75 nm, and interconnected internal porous network around 7-15 nm have been prepared by hybrid fabrication combining lithographic patterning and atomic dealloying. In addition to greatly enlarged surface area for ˜10× binding sites, a striking feature of NPGD is the high-density “hot spots” distributed across the entire particle, which is in drastic contrast to other plasmonic nanoparticles which feature primarily dipolar “edge” resonance. As a rule of thumb, target binding to hot spots will generate significant LSPR shift; those bind to “dark spot” will unlikely be detected. As a result, NPGD nanoarray provides superior sensitivity to target binding and less “blind spot”. Due to the large surface area, NPGD nanoarray has been shown to be superior photothermal heater than other plasmonic nanoparticles. The 3-dimensional porous network throughout the NPGD can been seen in a 45° view of shown in FIG. 3(e) where the nanoporous network can be observed clearly from the side of the nanodisk.

Fabrication techniques produce a NPGD nanoarray in a scalable fashion. Using nanosphere lithography (NSL), inch-scale NPGD nanoarray has been fabricated on silicon and glass substrate as shown in FIG. 4(a). NSL can produce highly regular NPGD nanoarray with nearly perfect hexagonal configuration and tunable-center-to-center distance that is much smaller than the disk diameter as shown in FIG. 4(b). Alternatively, using electron beam lithography (EBL), NPGD arrays in square configuration with precise diameter and center-to-center distance has been fabricated. FIG. 4(c) shows an array with 100 nm disk diameter and 100 nm spacing; FIG. 4(d) shows another array with 200 nm disk diameter and 100 nm spacing.

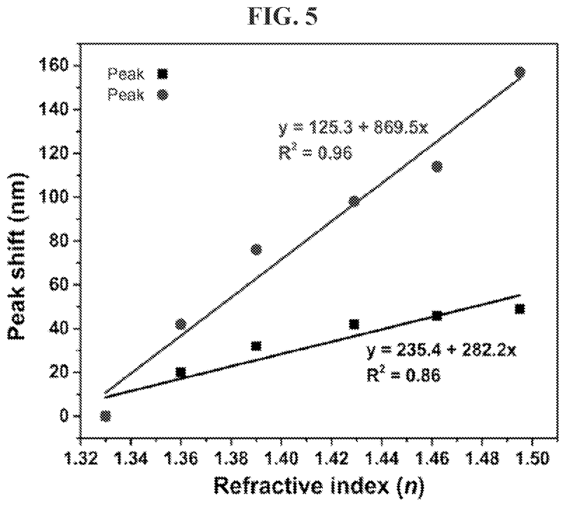

An advantage of NPGD is the high-density, 3-dimensionally distributed plasmonic hot spots identified across the entire NPGD. The NPG disk exhibits significantly high-density, uniform distribution of hot spot even when excited by linearly polarized light. In contrast, the non-porous disk has field concentrated on the left and right edges, aligning with the incident light polarization. In addition, the electric field around NPGD is significantly higher than that around the Au disk, thus the superior index sensitivity. Of particular significance is that NPGD can sense exosome binding regardless where the binding site is, whereas, a traditional non-porous disk can only sense exosome when it binds to the edges. Finally, the NPGD nanoarray produces some of the highest LSPR peak shift with respect to surrounding refractive index unit (RIU) change, reaching ˜900 nm/RIU (FIG. 5). The sensitivity is primarily due to LSPR interaction with index change no more than 50-100 nm away from the NPGD. The distance dependence makes NPGD a promising nanostructure for detecting exosome of size ˜30-150 nm over traditional SPR sensors. NPGD of diameter 130 nm and pitch 200 nm have been made.

High-sensitivity and -specificity sensing has been demonstrated with molecular sensors implemented on NPGD nanoarray for various target molecules such as DNA, malachite green, creatinine, rhodamine 6G, urea, dopamine, glutamate, cyanine 3, polycyclic aromatic hydrocarbons, tear glucose, urine acetaminophen, and the like, with concentration in the nano- to pico-Molar range (ppb-ppt), as well as cellular targets such as bacterial cells and spores with single unit sensitivity.

Additional MANSA surfaces with Au/Ag alloy and undercut gold can be utilized. Au/Ag alloy array exhibits symmetry-breaking induced nanoplasmonic mode splitting, resulting in a green peak at 540 nm. This has been employed for colorimetric detection of protein-protein interaction using low-cost camera (e.g., smartphone). Undercut gold array was fabricated by removing a large portion of the glass substrate beneath the gold nanodisks, which results in nanodisks of 350 nm diameter. The undercut array has superior index sensitivity.

### Example 1

Materials. Polystyrene beads of sizes 25 nm, 50 nm, 100 nm, 200 nm, 300 nm, 460 nm, and 750 nm were purchased from Sigma-Aldrich. Ethanol (200 proof) was purchased from Decon Laboratories, Inc. Gold sputtering target was purchased from ACI Alloys, Inc. Argon gas (99.999%) was used for RF-sputter etching.

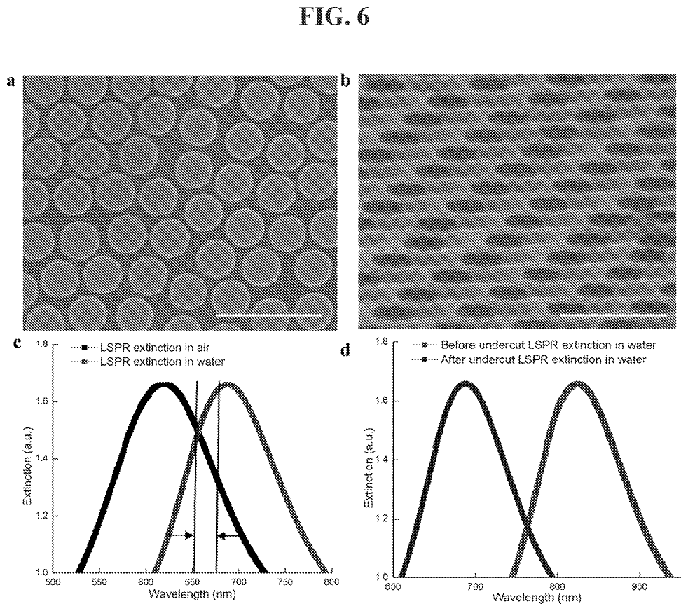

Fabrication of Nanodisk Array. Fabrication steps involve deposition of 2 nm of Titanium as an adhesion layer and then 80 nm of the gold film using E-beam evaporation. A monolayer of polystyrene beads of average diameter 460 nm was assembled over the gold film. The substrate was exposed to oxygen plasma etching to shrink the size of the polystyrene beads, followed by Argon Ion milling to etch away the uncovered part of the gold. The polystyrene beads were washed away via sonication. This generated a two-dimensional polycrystalline array of gold nanodisks with an average diameter of 360 nm with a pitch size (center-to-center distance) of 460 nm. FIG. 6(a) shows a SEM image of the exemplary nanodisk array. This gold nanodisk array was undercut in a buffer HF solution to partially remove the glass substrate beneath the disks. Due to the 100 nm edge to edge distance of nanodisk, it was difficult to image the undercut portion via SEM. Instead, a similar nanodisk array was prepared but with an edge to edge distance of 500 nm where the undercut portion was easily visible. This undercut nanodisk array is shown in FIG. 6(b). The LSPR extinction peak of the nanodisk array is at 620 nm in air and 690 nm in water, as shown I FIG. 6(c). FIG. 6(d) shows the LSPR peak blue shifted from 830 nm to 690 nm after the substrate undercut.

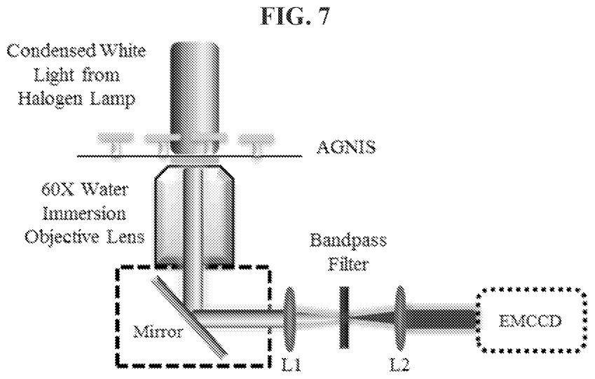

Optical Setup. White light from a tungsten-halogen lamp passes through a condenser (IX-LWUCD, Olympus) and illuminates the nanodisk array on an inverted microscope (IX71, Olympus). The transmitted light passes through an infinity corrected 60× water immersion lens with a 1.2 numerical aperture (UPLSAPO60XW, Olympus). The light exiting the side-port is relayed to an electron multiplied charge coupled device (EMCCD; ProEM 1024, Princeton Instruments) via a 4f system, with a bandpass filter with 650-670 nm passband (FB660-10, Thorlabs) at its Fourier plane. FIG. 7 shows a schematic for an exemplary optical system setup used in connection with this example. Other microscopy imaging modalities can be used, such as phase contrast, digital interference contrast, diffraction phase microscopy, polarization microscopy, quantitative phase imaging, and interference scattering microscopy.

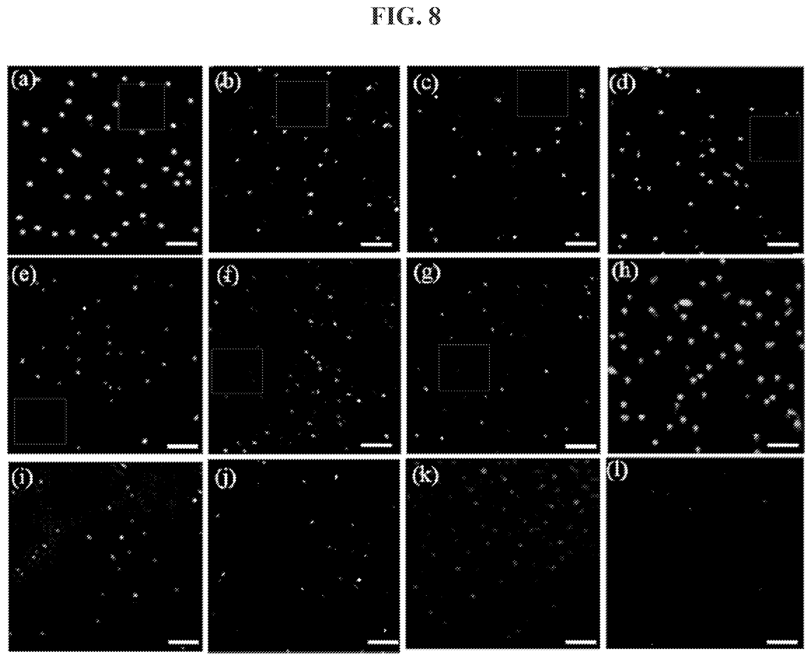

Nanoparticle Detection. Polystyrene (PS) bead sizes of 750, 460, 300, 200, 100, 50, and 25 nm were used to show the current imaging technique's performance in detecting tiny phase objects. A ratiometric image (“intensity ratio”) is obtained by dividing the image after nanoparticle settlement by the image without nanoparticles. For a control experiment a ratiometric image is obtained by dividing two images without nanoparticles, resulting in an image histogram with a mean of 1 and a standard deviation of 0.013, which is used as a baseline intensity ratio (IR) image. A threshold IR value was selected to be 1+3*0.013˜1.04. Assuming Gaussian statistics, an IR value larger than 1.04 indicates particle detection with a p-value<0.001. FIG. 8(a)-(g) show images of nanoparticles of all sizes after the thresholding process using the imaging techniques described herein. To compare the performance of the current plasmonic nano-aperture imaging techniques with standard bright-field microscopy, all PS bead sizes were imaged on a glass coverslip using the same setup, with images shown in FIG. 8(h)-(l). The current techniques provide images of detected beads of all sizes (FIG. 8(a)-(g)). In contrast, bright-field images (FIG. 8(h)-(l)) show a gradual decrease in intensity toward smaller bead size, and no detection was made for 100 nm (FIG. 8(l)) and smaller beads.

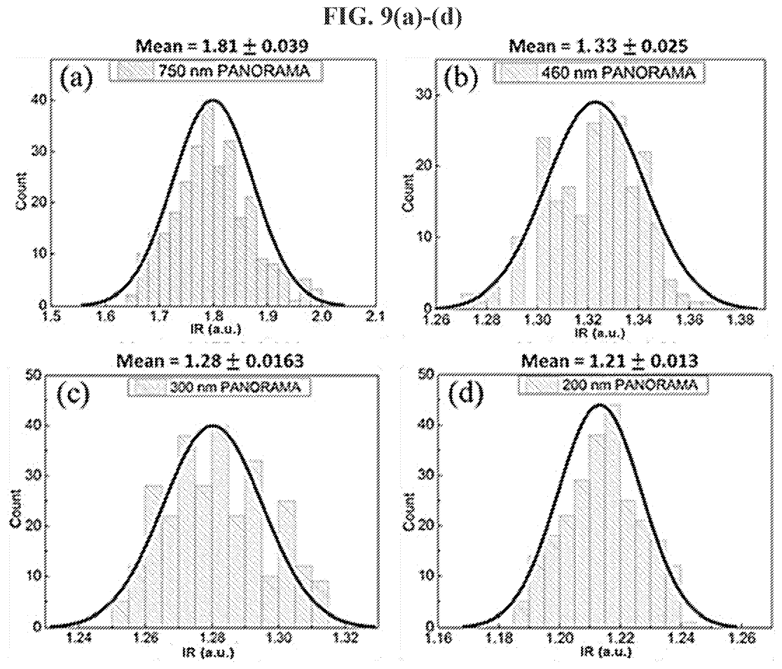

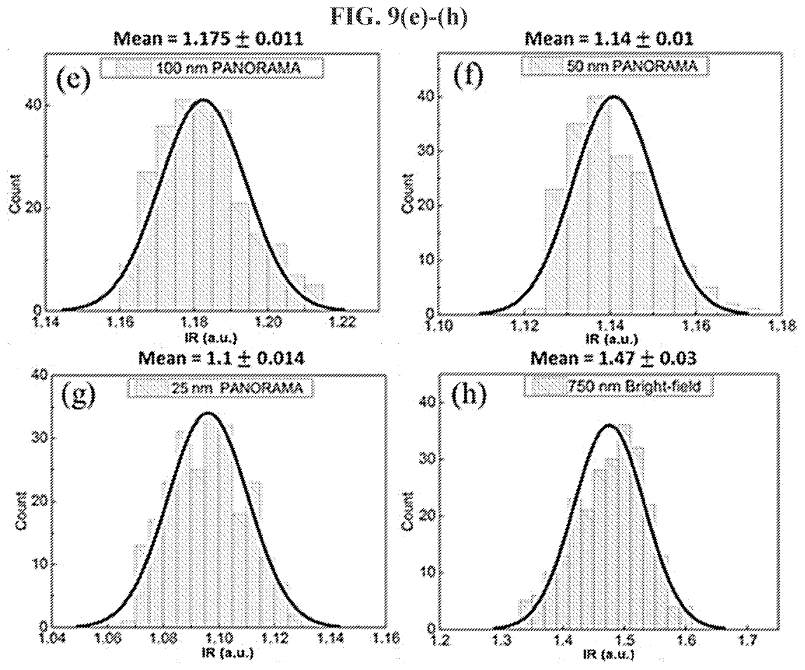

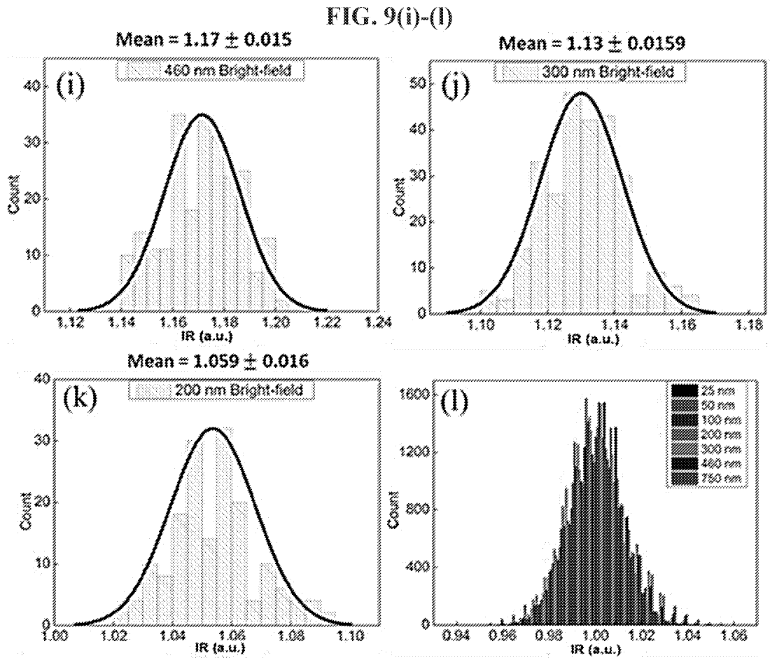

FIG. 9 shows a histogram analysis constructed from IR values recorded from all detected nanoparticles in FIG. 1 and control experiments. By applying a Gaussian fit, the mean IR values obtained using the plasmonic nano-aperture imaging techniques were 1.81, 1.33, 1.28, 1.22, 1.175, 1.14, and 1.1 for decreasing nanoparticle size (FIG. 9(a)-(g)), while the values from bright-field microscopy were 1.57, 1.17, 1.13, 1.06, 1.02, 1, and 1 (FIG. 9(h)-(k)). As mentioned earlier, an IR value smaller than 1.04 is considered not detectable. Considering the smallest PS bead size of 25 nm, the plasmonic nano-aperture imaging techniques provided an IR of 1.1 with sigma 0.014, which is well separated from the background histograms which centers at 1 with sigma 0.0131 (FIG. 9(l)), suggesting the potential of detecting even smaller particles or single molecules. Overall, the plasmonic nano-aperture imaging techniques were shown to detect PS bead sizes down to 25 nm, whereas bright-field microscopy cannot detect beads equal or smaller than 100 nm.

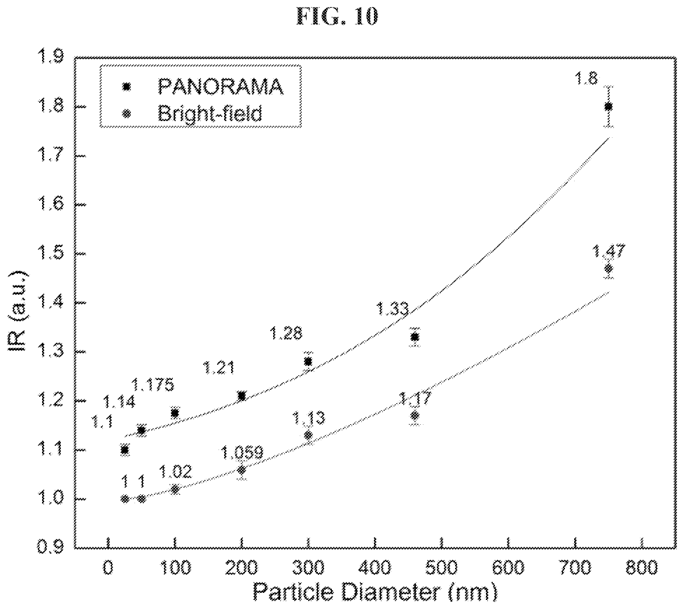

Considering the ability of the plasmonic nano-aperture imaging techniques to detect sizes reaching 25 nm, they cannot spatially resolve nanoparticle sizes below the diffraction limit. However, the mean and standard deviation of the histograms shows a correlation between nanoparticle size and IR where increasing nanoparticle size registers larger IR. FIG. 10 illustrates the image IR vs. nanoparticle size extracted from FIG. 9 for both plasmonic nano-aperture imaging (“PANORAMA,” squares) and bright-field microscopy (circles). The curves are provided for visual guidance. The averages and error bars were calculated from the points used to plot histograms in FIG. 9. It is observed that plasmonic nano-aperture imaging maintains a larger IR from 11%-22% among various nanoparticle sizes. It is remarkable to note that the plasmonic nano-aperture images' IR continues to decrease for nanoparticles smaller than 300 nm, suggesting that plasmonic nano-aperture imaging can provide size information beyond the diffraction-limited resolution.

It is worth noting that the current plasmonic nano-aperture imaging techniques have better contrast over IRIS for nanoparticles smaller than about 120 nm. For example, the current techniques provide a contrast of ˜14% for 25 nm PS nanoparticles while IRIS has a contrast<2%. In addition, the current techniques maintain ˜10% contrast for 25 nm PS nanoparticles but IRIS has already reached the noise floor. These results should not be too surprising. It is well known that LSPR has much better surface sensitivity than SPR and traditional interferometry.

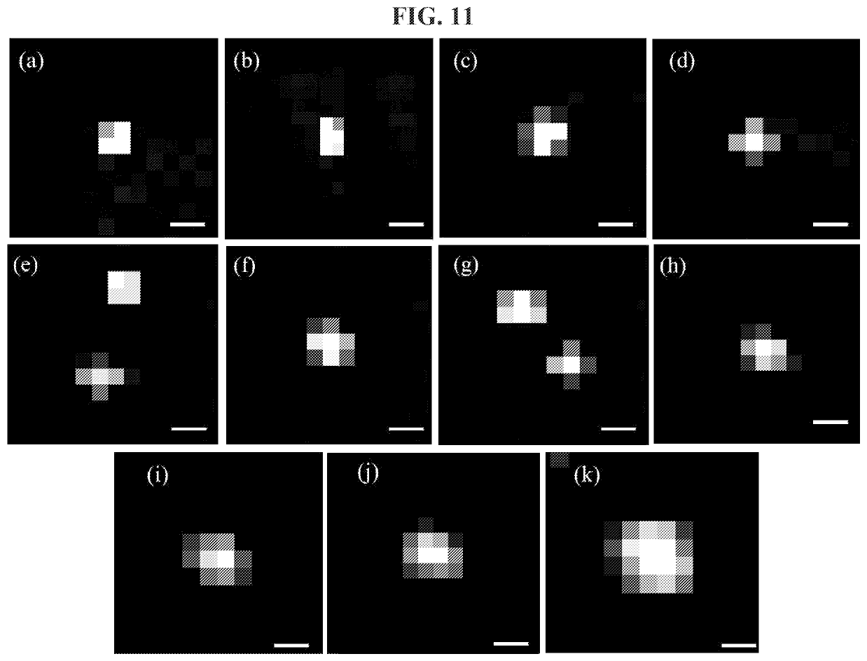

Plasmonic imaging nano-aperture size dependence on nanoparticle size. An interesting property of the current plasmonic nano-aperture imaging techniques lies in the nano-aperture size dependence on the nanoparticle size. The smallest achievable aperture size is roughly the gold disk diameter of 360 nm, which is comparable to the diffraction limited resolution (330 nm) and larger than the equivalent EMCCD pixel size (195 nm) at the sample plane. The gold disks can be made smaller than both the diffraction limited resolution and the camera pixel size. FIG. 11(a)-(d) show images of detected 25 nm PS beads, where a single pixel with the highest IR can be found to be accompanied with several surrounding lower IR pixels. The pattern varies according to the specific positions of the gold disk with respect to the EMCCD pixel grid, and to a certain degree the relative position of the PS bead on the gold disk. The minimum pattern size is equivalent to a patch of 2×2 pixels (FIG. 11(a)), but most detected nanoparticles appear in a patch of 3×3 pixels (FIG. 11(c)-(d)). PS bead sizes of 50, 100, 200, and 300 nm show similar patterns as 25 nm PS beads (FIG. 11(e)-(h)), suggesting the nanoparticle caused a single gold disk to shift in most cases. When the nanoparticle size becomes comparable to the gold disk, e.g., 300 nm PS beads, the pattern can occasionally appear as a slightly larger patch (FIG. 11(i)). A plausible explanation is as the nanoparticles become larger, the probability increases for a single nanoparticle to causes LSPR shift for the adjacent disk(s). Nano-aperture across multiple gold disks becomes more apparent for larger nanoparticles as shown in FIG. 11(j) for 460 nm and FIG. 11(k) for 750 nm, respectively. The sampling density can be improved by higher magnification and smaller camera pixels, which will be investigated in the future.

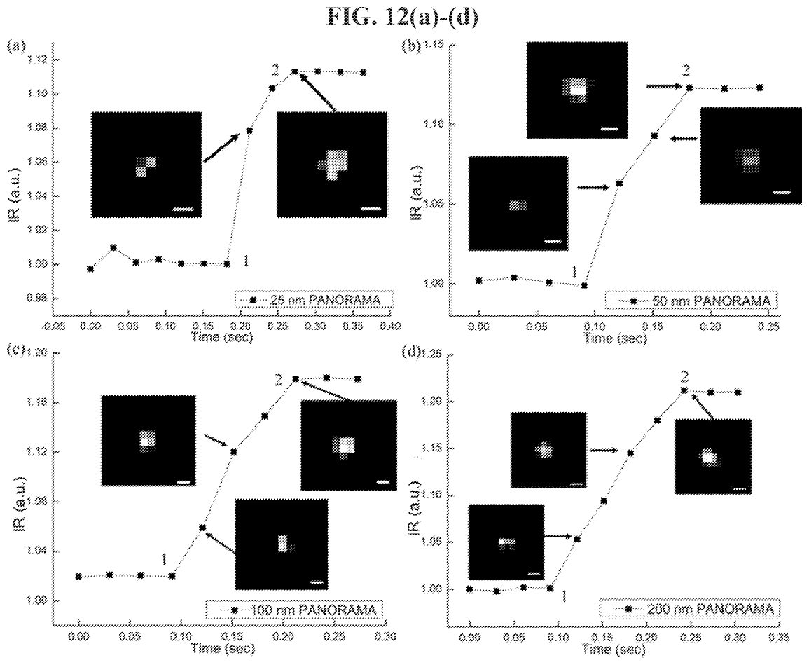

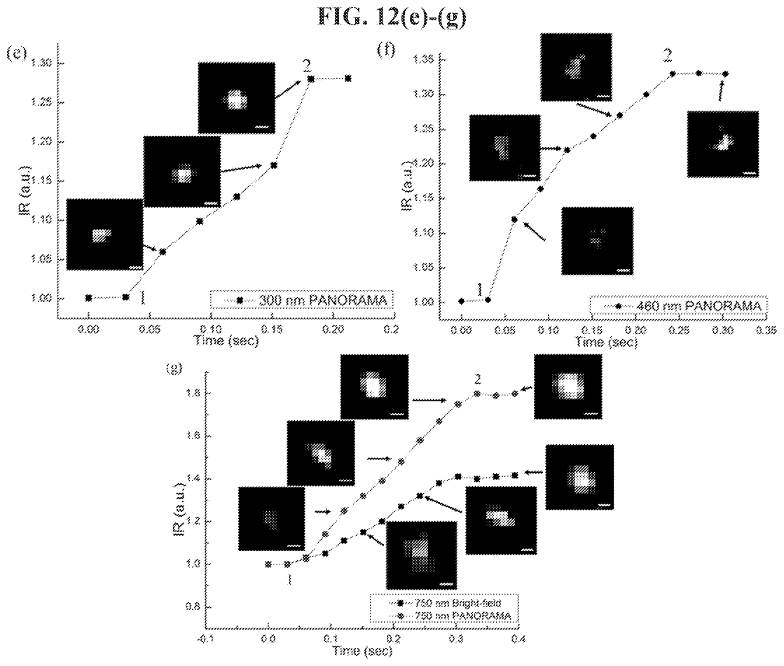

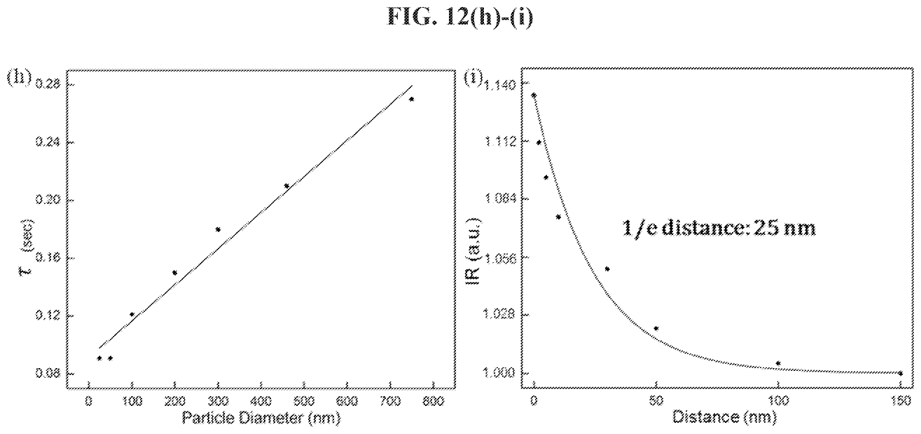

Interpreting nanoparticle to surface distance by dynamic monitoring. A key strength of the current plasmonic nano-aperture imaging is its outstanding light throughput, which enables dynamic imaging at millisecond timescale. To demonstrate this experimentally, dynamic imaging was performed at 33 Hz frame rate and 10 ms integration time per frame during nanoparticle settling. The frame rate was limited by preventing the camera from overheating. Similar IR values have been obtained from 25 nm PS beads with as little as 1 ms integration time, suggesting the potential of reaching kHz frame rate. FIG. 12(a)-(g) shows the IR values of various size PS beads as they settle on the nanodisk array. Nanoparticle settling time (r) can be defined as the travel time between the nanoparticle is first detected and IR plateau (complete settlement) as shown in FIG. 12(a)-(g), suggesting a trend of increased r with increased nanoparticle size. FIG. 12(h) shows particle settlement time (r) vs. particle size. r was calculated from the points labeled 1 to 2 for all particle sizes in FIG. 12(a)-(g). Assuming the settling speed does not strongly depend on size, the larger r for larger nanoparticles suggests that they can be detected farther away from the nanodisk array surface. The plateau IR values for all particle sizes agree well with the previously established values after nanoparticles settlement (FIG. 10).

To gain more insight on the surface sensitivity of the current imaging techniques, finite difference time domain (FDTD) simulations were performed where a 50 nm PS nanoparticle was placed at various distances from the nanodisk array. As shown in FIG. 12(i) the IR of the particle increases as the nanoparticle becomes closer to the nanodisk array with a lie distance of 25 nm. Interpreting the 50 nm nanoparticle result in FIG. 12(b) in the light of the simulated results, the nanoparticle was outside the detectable distance (˜100 nm) from the nanodisk array surface prior to t˜99 msec, after which it moved toward the nanodisk array and arrived at the surface at t˜190 msec. As shown previously, a 50 nm PS nanoparticle did not provide detectable light scattering in the bright-field imaging mode, suggesting the settling process was imaged entirely by the plasmonic nano-aperture technique. In contrast, larger nanoparticles (e.g., >100 nm) can provide detectable light scattering as long as the nanoparticle enters the imaging system depth of focus (DOF)˜+−413 nm. In other words, larger nanoparticles can be first detected when they are significantly farther away from the nanodisk array surface entirely due to its own light scattering. In the case of 750 nm nanoparticles (FIG. 12(g)), it first entered the DOF at t˜66 msec, then moved toward the nanodisk array surface and arrived at t˜333 msec. It is remarkable that plasmonic nano-aperture imaging contributes additional IR even for larger nanoparticles as shown in FIG. 12(g). The time for the 750 nm PS bead to settle on both the glass coverslip and the nanodisk array's surface is the same, however, the slope of the IR vs. time curve for plasmonic nano-aperture imaging is significantly higher than that of the standard bright-field image, indicating higher distance sensitivity.

These results suggest that the r during nanoparticle settlement can provide an additional means for nanoparticle sizing. They also suggest that it is possible to monitor the distance between the nanoparticle and the nanodisk array and obtain velocity with a resolution far better than the diffraction limit provided careful calibration and higher imaging speed.

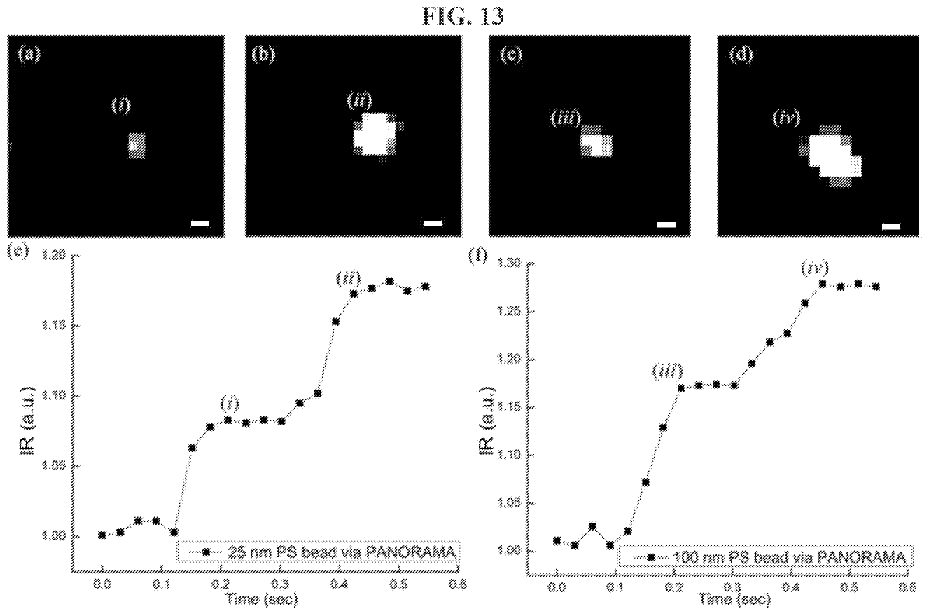

The dynamic imaging capability can be further exploited to count nanoparticle clusters with interparticle distance smaller than the diffraction limit. Counting is achieved by monitoring the IR fluctuations over time. This can be seen in FIG. 13(a) where a 25 nm PS bead was initially settled on the surface at time i, followed by a second bead settling into the same region at time ii and forming a cluster as seen in FIG. 13(b). The process is evident by monitoring the IR over time in FIG. 13(e) where the first bead shows a typical IR value for a single 25 nm PS bead. At time ii, the IR jump indicates the settlement of a second particle. The same behavior can be seen for a 100 nm PS bead in FIGS. 13(c), (d), and (f).

Plasmonic nano-aperture label-free imaging has been demonstrated as a novel nanoparticle imaging technique. Its basic principle stems from virtual nano-apertures modulated by ultra near-field refractive index. Instead of measuring the light scattered by the nanoparticle, which diminishes dramatically with reducing size, plasmonic nano-aperture imaging detects both the scattered and unscattered light modulated by the nano-aperture, thus enjoys unprecedented sensitivity. Plasmonic nano-aperture imaging addresses several limitations in existing SPR and LSPR imaging techniques by providing large lateral imaging fill factor and extended longitudinal imaging range while achieving diffraction-limited imaging resolution. Its system configuration is identical to a standard bright-field microscope using a trans-illumination tungsten-halogen lamp instead of lasers or other high-intensity light sources, and a single camera. The bright-field approach provides much higher light throughput for dynamic imaging at the millisecond time scale compared to dark-field microscopy that suffers low light throughput. Plasmonic nano-aperture label-free imaging can size a single nanoparticle down to 25 nm, dynamically monitor single nanoparticles approaching the plasmonic surface, and count individual nanoparticles in a cluster. These imaging techniques can provide new capabilities in label-free imaging and single nanoparticle analysis. More importantly, molecular imaging can be envisioned with a functionalized nanodisk array surface for single biological nanoparticle analysis including extracellular vesicles such as exosomes.

### Example 2

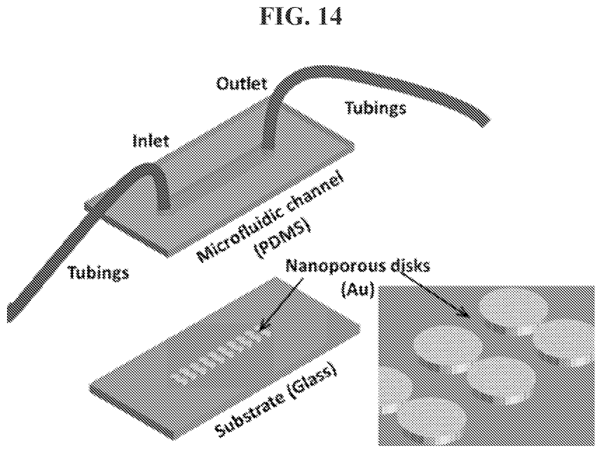

Monolithic integration of NPGD nanoarray and microfluidics. The fabrication process flow shown in FIG. 1(a) was used to fabricate a single-well MANSA as shown in FIG. 14. The microfluidic chip has single inlet/outlet that are connected to precision pumping systems that can produce a flow rate between 1-50 μL/min without pulsation. The microchannel was proven to be properly sealed without leakage. The integrated NPGD nanoarray was shown to be a high-performance nanoplasmonic sensing surface by a variety of optical techniques including LSPR shift, surface-enhanced Raman spectroscopy (SERS), surface-enhanced fluorescence (SEF), and surface-enhanced near-infrared absorption (SENIRA). This device provides a solid foundation to scale up into a 3×3 microwell matrix.

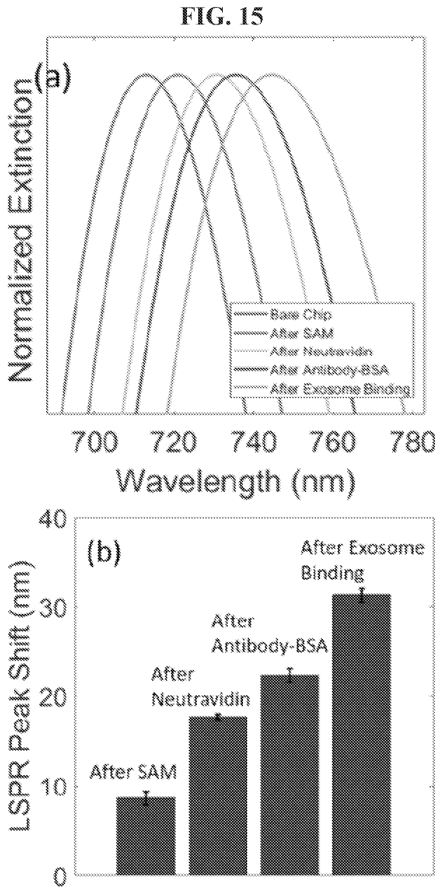

Surface functionalization for specific CTE enrichment and detection. To demonstrate the proposed selective enrichment and label-free detection, the MANSA was functionalized with antibodies that can recognize up-regulated surface antigens on cancer exosomes such as CD9, CD63, and CD81. The functionalization and linking steps are generally shown in FIG. 1(b). Briefly, a thiol-poly(ethylene-glycol) (PEG)-biotin self-assembled monolayer (SAM) is first coated onto the nanoarray surface by incubating overnight at 5 mM. Neutravidin was then introduced, followed by the biotin-antibody and bovine serum albumin (BSA) passivation. To ensure the control of highly precise surface functionalization, in situ LSPR shift was monitored by spectroscopic imaging system. FIG. 15(a) shows the LSPR extinction spectra after successive steps of surface functionalization. The LSPR peak shifts are summarized in FIG. 15(b), where a total of ˜22 nm red-shift is observed up to after the BSA passivation. CTE was subsequently flowed by and an additional ˜10 nm red-shift was observed. In a negative control experiment where antibodies were intentionally left out from the surface functionalization steps while everything else identical, the additional shift due to exosome is ˜2 nm. Therefore, the data show that exosomes are indeed detected on the MANSA.

Bio-particle manipulation is a powerful technique as shown in recent advances in acoustofluidics. Due to its highly tunable LSPR peak position, low-power generation of nanoplasmonic microbubbles can be implemented on MANSA using a 785 nm laser with power well under 5 mW. The microbubble can concentrate nanoparticles in the microfluidic chamber to the bottom nanodisks surface in a size selective manner By modulating the laser at different frequencies, smaller nanoparticles tend to be concentrated preferentially while larger nanoparticles and microparticles are not. It is remarkable that at even lower power (<1 mW), nanobubbles can be formed within the internal porous space of the NPGD without the formation of any visible microbubble. This occurs because when the water inside the porous voids of NPGD is replaced by gas with lower refractive index, LSPR blue-shift results in more attenuation and thus a darker area within the illuminated spot compared to the outer unilluminated area. The nanobubble has two critical advantages: (1) to reduce any thermal damage to the functionalized surface; (2) it concentrates exosomes gently from the suspension toward the MANSA surface. Exosomes cannot be observed when >˜200 nm from the surface. A large number of 50 nm nanoparticles (mimicking exosomes) was shown to be concentrated on the MANSA surface after operating the nanobubble for 5 minutes.

As individual exosomes bind to the antibody-functionalized nanodisks surface, it increases the local refractive index from the ambient (1.33 for aqueous) tob>1.4. Such refractive index jump causes the LSPR to red-shift. The magnitude of LSPR shift can be correlated to the number of exosomes. A narrow-band filter-based system is used for dynamic nanoplasmonic imaging at diffraction-limited resolution. In other words, the MANSA surface is virtually partitioned into numerous sensors with 350×350 nm² in size. For an array size of 1 mm², this corresponds to >1 million sensors. Various data acquisition/processing conditions optimize sensitivity and signal-to-noise ratio. The bound exosomes will be subsequently imaged by a spectroscopic imaging system which generates a complementary LSPR peak map at near diffraction-limited resolution. When nanosphere lithography (NSL) is employed, on average<4 nanodisks will be monitored on the 350×350 nm² footprint, whereas when electron-beam lithography (EBL) is employed, a single nanodisk will be monitored. Other large-area lithography techniques such as nanoimprint lithography, colloidal lithography and advanced photolithography can also provide similar large-area patterning. In both scenarios, gradual LSPR shifts will be detected for counting. Since LSPR depends on the incident light polarization, super-resolution is possible by gating the polarization and incidence angle of the excitation light, as demonstrated by us and others. After CTE binding, a second spectroscopic imaging system is employed to acquired full extinction spectra at 500×500 nm². LSPR shift map at this resolution will then be derived to complement the dynamic imaging data.

Surface-enhanced multiplex molecular beacon design and implementation: Current exosomal D/RNA profiling does not have sufficient sensitivity required to detect minimum residual disease (MRD). To overcome this, molecular beacon (MB) hybridization based probes are used which have been demonstrated to have excellent sensitivity, specificity with multiplex. MB has been demonstrated to detect miRNA and mRNA in whole exosomes, but single exosome detection has not been shown. MBs are single-stranded DNA (20-40 mer) designed to fold into a stem-loop hairpin configuration. The loop is designed to be complementary to the target sequence. The 5′ end is attached with a fluorophore and the 3′ end a fluorescence quencher. When the MB probe is in the hairpin configuration, the fluorophore is quenched. However, the MB probe can hybridize with a target sequence and form the double-stranded structure, where the fluorophore is now far away from the quencher so it can fluoresce. MB probes have been proven to be sensitive to detecting single nucleotide polymorph and single point mutation. However, MB fluorescence can be too weak to detect from single CTE. Our approach is to enhance the MB fluorescence intensity of MB by LSPR, a mechanism known as metal-enhanced fluorescence (MEF) or surface plasmon-enhanced fluorescence (SEF).

MEF/SEF occurs when fluorophores are within nanometric proximity from metallic surfaces, e.g., 10-50 nm. The process has been proved to be advantageous in various ways: faster spontaneous emission processes, increased quantum yields, improved fluorophore photostability, and shorter fluorescent lifetimes. MEF/SEF studies have gained the attention of the chemical and biological research community due to the possibility of detecting analytes at extremely low concentrations with fluorescence enhancement factors up to 500. When the exosomes are immobilized on the nanodisks, a significant portion of it is within the region of enhanced electric field, causing bright MB fluorescence. MB probes targeting 3 oncogenic miRNA: miR-21, miR-17-92 cluster, and miR-221/222 paired with different fluorophores can be designed. Since all the nanoplasmonic measurements and concentration/isolation are performed in the near-infrared (NIR) wavelength range (700-950 nm), the low energy photons run into little risk of phototoxicity and photobleaching, and would not interfere with any fluorescence measurements. Several suitable fluorophores for the molecular beacons include: Cy 3, Cy 5, BV421, 488, 647, 480, and 568.

Enhance delivery of molecular beacons into exosomes: To facilitate the MB probes to enter exosomes, three approaches can be used: (1) using liposome packaging as a proven delivery vehicle; (2) using nanoplasmonic heating to increase cross-membrane permeability; and (3) using nanodisks as contact electrodes for electroporation. Due to the lipid bilayer structure of exosomal membrane, using similarly-sized liposome as delivery vehicle has been demonstrated for messenger RNA. MB embedded liposomes in the size range of 100 nm are prepared for facile delivery. In addition, using nanoplasmonic heating, light-modulated membrane permeability can be explored to enhance MB probe delivery into exosomes. Moreover, by fabricating the nanodisks array on a transparent conductor such as indium tin oxide (ITO), they can be electrically connected into one bottom electrode. Using one of the microfluidic inlet tubing as the other electrode, a current loop can be established. Electroporation has been employed for cellular poration, but not for exosomes. However, since the membranes are identical, it should work effectively on exosomes.

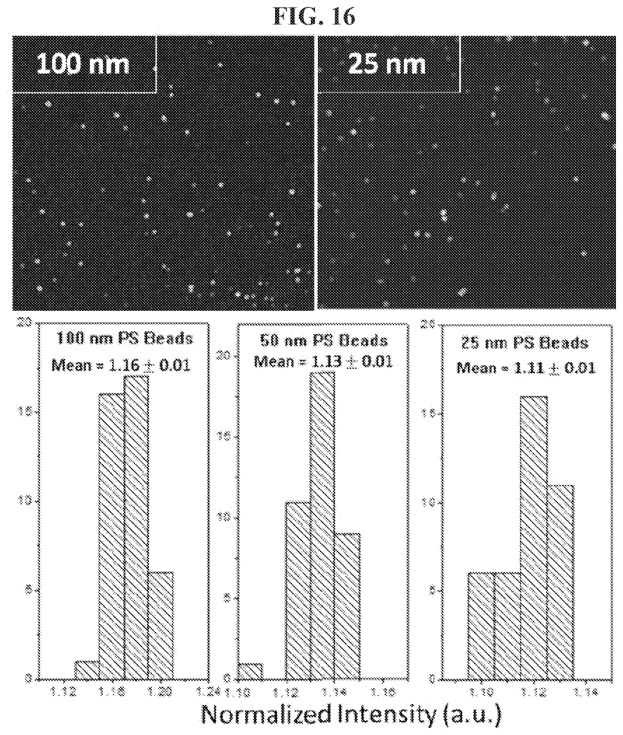

High-resolution imaging of exosome binding onto MANSA: Single exosome counting by dynamic nanoplasmonic imaging: A bright-field imaging system has been developed to monitor the intensity within a narrow-band of the LSPR peak. When an exosome binds to MANSA, an intensity jump can be detected due to the LSPR red-shift. As shown in FIG. 16, the sensitivity of this system is remarkable—polystyrene nanoparticles down to 25 nm can be readily detected. Because the scattered light intensity scales with (particle diameter)⁻⁶, dielectric nanoparticles<100 nm are challenging to detect in the bright-field. In these results, the contrast was 1.16, 1.13, and 1.11 with respect to the bright-field background of 1 for 100, 50, and 25 nm polystyrene (PS) nanoparticles, respectively. The displayed images were background subtracted and thus appear like “dark-field” to accentuate the nanoparticles, however, the raw images were acquired in bright-field transmission mode without special dark-field imaging optics.

In the transmission mode, MANSA can be understood as an attenuating mask due to LSPR absorption/scattering. The local LSPR red-shift due to nanoparticle near the MANSA reduces the attenuation and allows more unscattered light to pass through. Therefore, the high sensitivity of this scheme comes from a virtual nano-aperture being opened when nanoparticles are on the surface. Since the unscattered light is order of magnitude stronger than the scattered portion, a much better contrast can thus be observed. This technique has a key advantage in surface coverage (˜100%) over traditional dark-field imaging on sparse plasmonic arrays with low surface coverage (misses lots of exosomes), low optical efficiency, and low saturation limit and dynamic range. The present imaging techniques also work in the reflective mode (i.e., “epi”) because the primary reason for the increased transmission is reduced light scattering by the nanodisks. Thus, a darker pattern (i.e., lower light intensity) due to reduced scattered reflection is expected when the technique is implemented in the reflective mode. The present imaging techniques also work with total-internal-reflection (TIR) excitation, similar to SPR imaging.

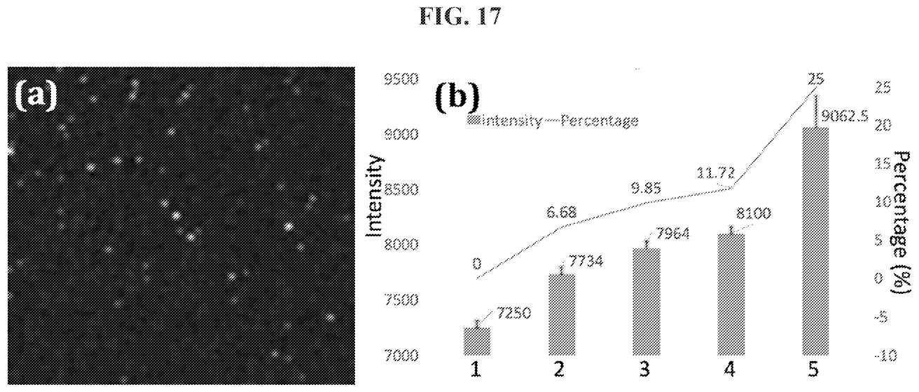

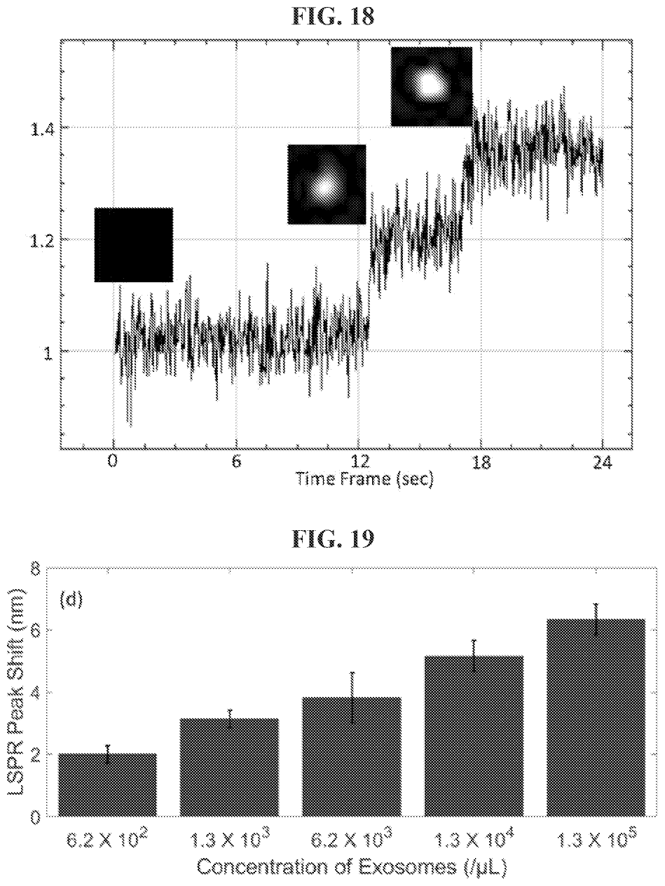

Exosomes extracted from culture media of H-460 lung cancer cells have been analyzed. A size distribution histogram shows a peak at ˜125 nm and mean ˜150 nm. A dynamic imaging approach was employed to monitor the MANSA surface through successive functionalization steps until exosome binding (Step 5 in FIG. 17(b)). The endpoint image is shown in FIG. 17(a) where the detected single exosomes are highlighted. The intensity changes going through all steps are shown in FIG. 17(b), where a general trend of intensity increase is observed as expected by successive red-shift. It is worth noting that the final exosome binding (Step 5) generates a remarkable intensity jump compared to prior functionalization steps. A noteworthy capability of the dynamic imaging technique is it can resolve successive binding events of different CTE within a single resolution unit. As shown in FIG. 18, CTE #1 and #2 bind at 12s and 18s, respectively. Both events are detected as intensity jumps.

LSPR peak map by spectroscopic imaging: To obtain the full spectroscopic image in (x-y-λ), a high-resolution, spectroscopic imaging system was used to acquire large-area mapping of LSPR shifts at ultrahigh precision and reproducibility: The shot-to-shot reproducibility of the system at 500×500 nm² spatial resolution is 0.2 nm. In other words, LSPR shifts larger than 0.2 nm can be reliably quantified. This system was used to monitor the surface functionalization process as well as exosome capturing on undercut gold nanoarray. The maximum LSPR shift was about 16 nm and spatial heterogeneity was observed, likely due to non-uniform coverage of exosomes. A 130 nm diameter nanodisk array with 70 nm edge-to-edge gaps will virtually guarantee, on average, only one exosome can bind to one nanodisk. Using the LSPR peak mapping system, a series of experiments were performed using different exosome concentrations with results shown in FIG. 19. The results suggest that as few as 600 exo/μL can be detected as 2 nm shifts. The system has a reproducibility of 0.2 nm, so 2 nm is far above the noise/error floor. In addition, any shift contributed by non-specific binding has already been excluded from the results, thus the resulted shifts can be confidently attributed to H-460.

Because the data are acquired after all the exosomes had already bound, a single exosome cannot be resolved if a cluster is formed within the resolution unit (˜500 nm). However, superresolution by detailed analysis of the LSPR intensity, peak shape/width, and shift can be considered. Using finite-difference-time-domain simulation, many possible scenarios exist for exosome binding on NPG disk. For the 8 different binding situations, the LSPR shifts results suggest that the magnitude of LSPR shift indeed correlates well with the number of binding, but a minor dependence on binding location with respect to the NPG disk cannot be ignored. This approach is furthered by optimizing the system and developing better analytical techniques to improve exosome counting resolution. Another strategy is to find the optimal binding conditions such that statistically only 1 exosome can be expected on each imaging resolution unit. A third strategy is to vary polarization and incident angle to pinpoint the exosome binding location. Exosomes can potentially be resolved by deconvolution of the LSPR spectrum.

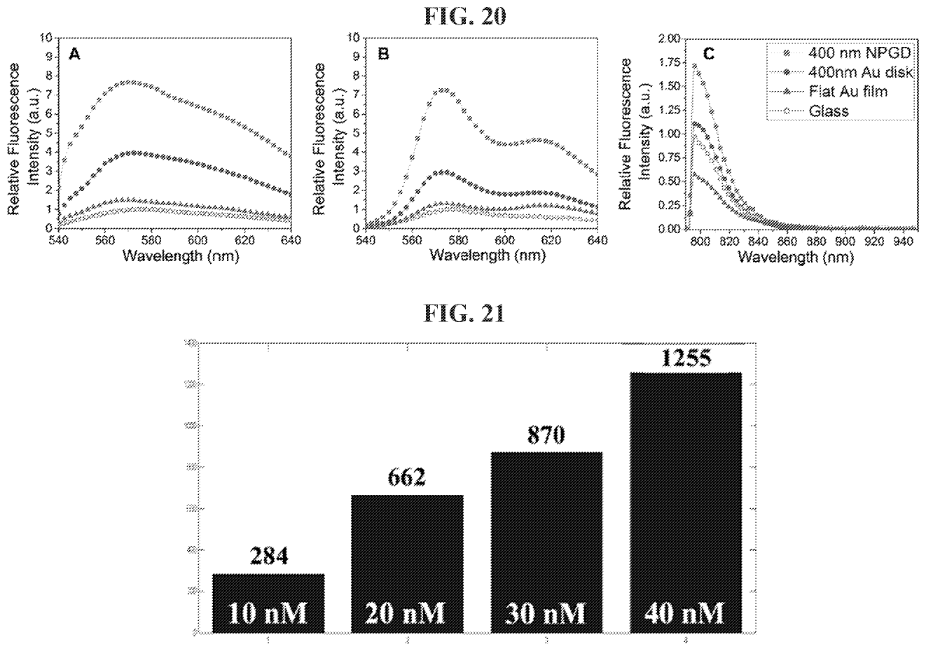

A dual-mode nanoplasmonic molecular beacon/sentinel platform on NPGD nanoarray has been implemented for label-free detection of breast cancer gene (HER2, or ERBB-2) [63, 65]. By immobilizing an MB hairpin loop DNA with 5′-thiol and 3′-cyanine (Cy3) fluorophore on NPGD, the metal acts as effective fluorescence quencher. Once the MB probe hybridizes with the HER2 sequence, the Cy3 is relocated to −8-10 nm away from the gold surface, where surface-enhancement is strong. The technique can achieve a LOD of 36 molecular beacon molecules. As shown in FIG. 20(a)-(c), 3 different common fluorophores (R6G, Cy3, IRDye) were used to compare fluorescence intensity on four different substrates. The results show that MANSA produces the highest enhancement. Significant SEF from quantum dots on NPGD nanoarray has also been demonstrated. The results provide strong support for the proposed enhanced fluorescence MB inside immune-captured exosomes. An exemplary MB with Cy3 fluorophore and a sequence complementary to miR-21 has been designed. Results from solution mixture measurements are shown in FIG. 21, suggesting the hybridization-triggered MB fluorescence bears an approximately linear relationship with miR-21 concentration. The MB will be delivered into exosomes captured on MANSA.

Further enhanced delivery of molecular beacons into exosomes. The MBs enter into exosomes that are immobilized on the MANSA. The electric field enhancement can extend at least 30 nm into exosome. In other words, a very substantial portion of the MB probes inside the exosome can be significantly enhanced by the nanoplasmonics, which will make detection of small number and even single CTE possible. Although exosome membrane is similar to that of cells, it lacks active transport mechanisms like endocytosis. Together with the detailed single exosome counting and location registration by the dynamic nanoplasmonic imaging, how many exosomes contribute to the MB fluorescence from within a resolution unit can be estimated, thus rendering the fluorescence measurements for single exosomes.

### Example 3

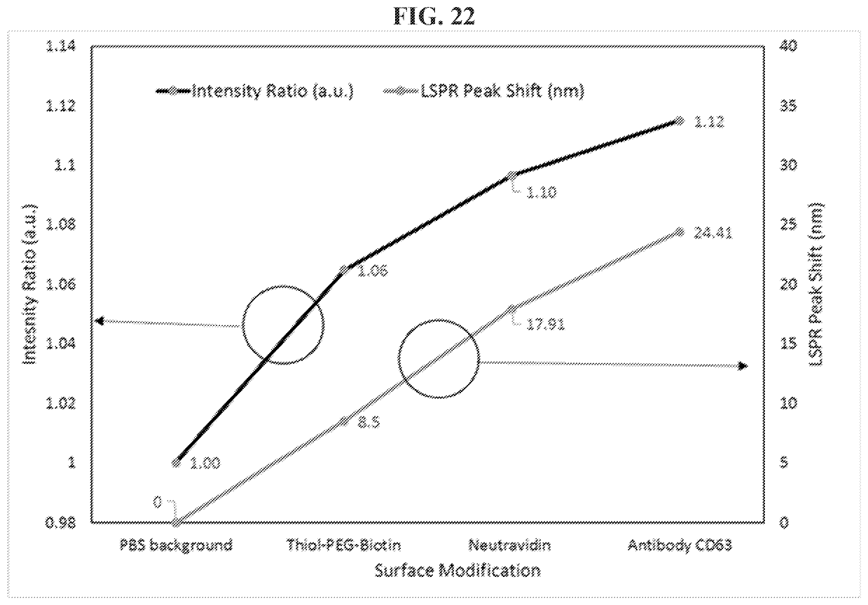

An exemplary nanodisk array substrate was subjected to various surface modification steps to functionalize the surface with antibody CD63. As a confirmation for surface functionalization, the surface was monitored for IR changes via the current imaging techniques and LSPR absorption peak red shift. FIG. 22 shows IR and corresponding LSPR peak shifts recorded after different steps of the surface functionalization. As shown in FIG. 22, surface modifications include a monolayer of Thiol-PEG-Biotin, followed by the binding of neutravidin and biotin-CD63 antibodies which have all shown a gradual increase of both Intensity Ratio (IR) and a constant red shift of LSPR absorption peak (vertical axis to the right). The multiple-step surface functionalization followed a protocol published in Nature biotechnology 32.5 (2014): 490-495 for exosome detection. Every step of the surface modification recorded a change of both IR via plasmonic nano-aperture imaging on the surface and recorded an LSPR peak shift. The binding of Thiol-PEG-Biotin changed the surface IR to 1.065 and an LSPR extinction peak shift of 8.5 nm compared to the bare background. The addition of the Neutravidin monolayer further increased the IR to 1.095 and increased the LSPR peak shift to 17.9 nm. Finally, adding the antibody recorded an IR of 1.125 and an LSPR peak shift of 24.4 nm compared to the bare nanodisk array surface. The IR changes correlate well with the LSPR shifts.

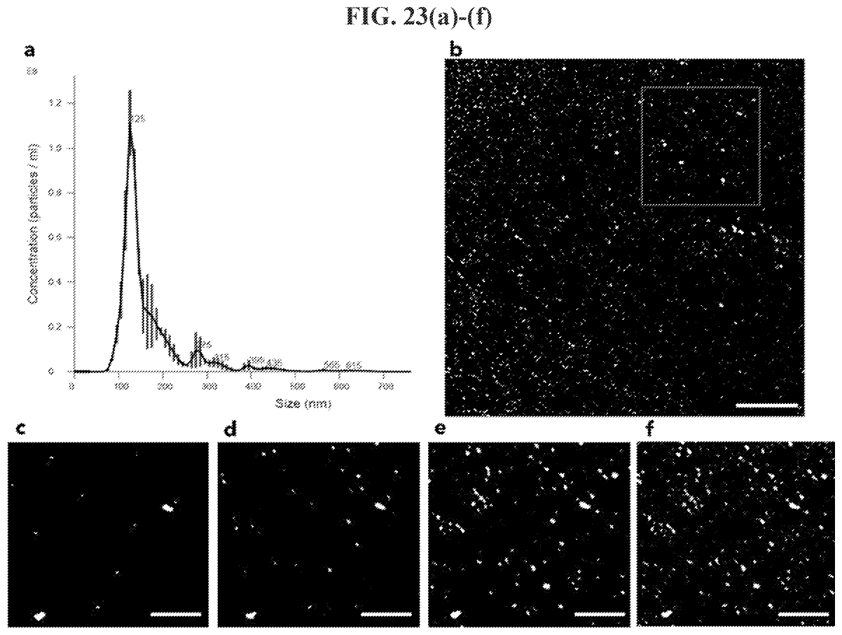

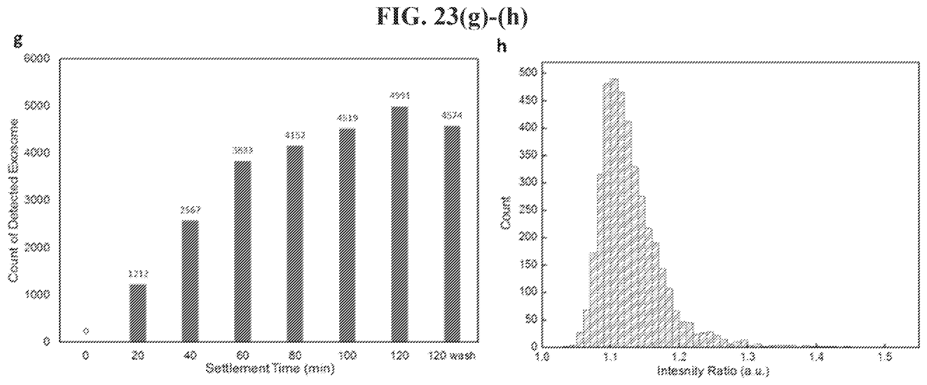

The exosomes used were from cell line H460 and show a size distribution of between 50 to 250 nm on average with the highest majority at 125 nm. FIG. 23(a) shows size distribution of the exosomes measured via Nanosight particle tracking. The solution of exosomes used in this experiment contained 2×10⁵ exosome/μl and was placed on the functionalized nanodisk array for 2 hours to provide sufficient time for exosome settlement on the surface. After 2 hours the surface was washed with PBS 1×, repeated 5 times, to remove any excess exosome with non-matching surface protein to the used antibody. FIG. 23(b) shows an image of detected exosomes collected using the plasmonic nano-aperture techniques after 120 min of settlement after undergoing a washing process to remove unbound exosomes. As shown in FIG. 23(b), the washed surface compared to the bare functionalized surface showed an intensity increase indicating the detection of exosome via the current imaging techniques. Furthermore, monitoring the marked region in FIG. 23(b) at various time stamps showed a gradual increase of detected exosomes while showing the detected exosomes at previous times. FIG. 23(c)-(f) shows exosome detection in the marked location after (c) 20 min, (d) 40 min, (e) 80 min, and (f) 120 min of particle settlement time before washing. The number of exosomes increased and also recorded the exosomes detected at a previous time. FIG. 23(g) shows the number of detected exosomes over time and after washing, in a 100 μm×100 μm image. The total number of exosomes settled on the surface peaked at 4991 which decreased to 4574 after washing. Overall, 91% of exosomes remained immobilized on the surface after washing with PBS. The IR distribution of the exosome was also extracted which showed an average of 1.13±0.05 and an overall distribution similar to the Nanosight size distribution. FIG. 23(h) shows a compiled histogram of the detected exosome IR from FIG. 23(b).

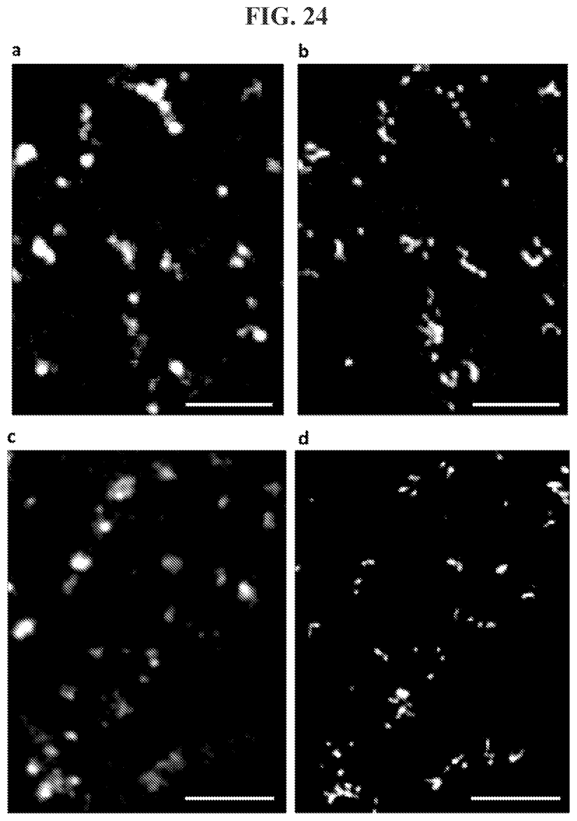

The exosomes were also labeled with PKH67 fluorescent dye to confirm their presence. FIGS. 24(a) and (c) show fluorescence images of exosomes settled on the functionalized nanodisk array surface, while FIGS. 24(b) and (d) show the plasmonic nano-aperture images of detected exosomes shown in FIGS. 24(a) and (c). Both imaging systems reveal a well-matched image of the detected exosome.

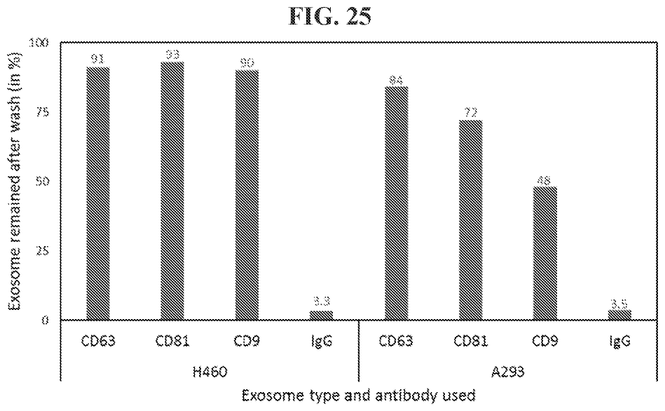

The surface protein composition may vary between cancerous and non-cancerous exosomes and may provide insight into tumor progression. To determine protein expression patterns associated with cancer, the amount of remaining immobilized exosomes were examined after the washing via the current plasmonic imaging techniques. FIG. 25 shows profiling of exosome populations (cancerous H460 and non-cancerous 293A) defined by surface protein (CD63, CD9 and CD81) obtained by monitoring the percentage of exosomes remaining on the surface of the nanodisk array after washing. Antibodies CD63, CD81, CD9, and IgG were tested on two populations of exosomes one extracted from non-cancerous 293A cell line and the second from cancerous H460 cell. The surface proteins CD63, CD81, and CD9 were well expressed on H460 exosomes wherein in all 3 cases, the percentage of remaining exosomes remaining after wash was above 90%. Furthermore, CD63 proteins showed minimal change on 293A exosomes. However, both CD81 and CD9 proteins showed a lower expression on non-cancerous 293A exosomes with CD9 showing the most prominent change. As a final step, the nanodisk array surface was functionalized with IgG as a control showing. The control experiment showed a significantly lower percentage of captured exosomes with 3.3% and 3.5% of exosome capture for H460 and 293A exosomes, respectively. Overall, studies show CD63 as a general surface protein for exosome which can be beneficial to immobilize larger quantities of exosome on a functionalized surface. As for CD9 proteins, it showed the most potential as a proteomic biomarker for cancer progression.

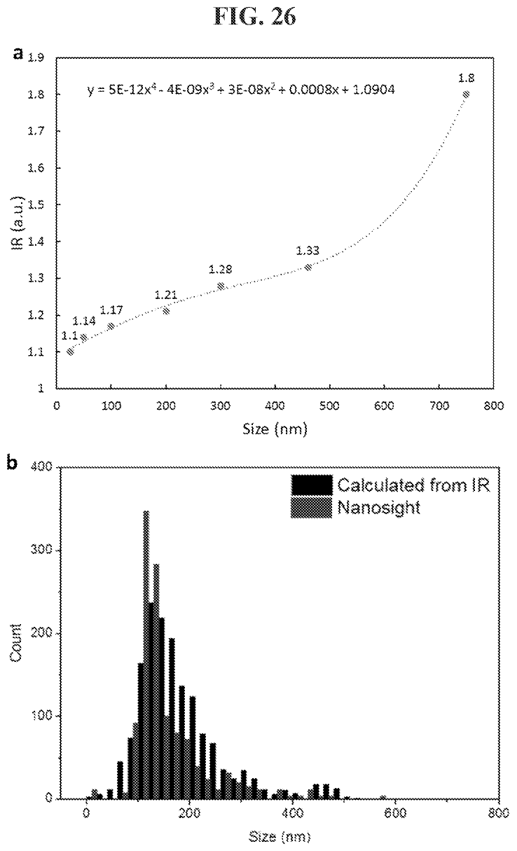

A trend can also be observed between particle size and recorded IR as seen in FIG. 26(a). FIG. 26(a) shows a calibration curve of IR to size using polystyrene beads. As seen in FIGS. 23(a) and 23(h), the majority of exosomes are in the range of 125 nm and an IR of 1.13 indicating a similar trend between the 2 figures. The calculated size distribution from IR was plotted and compared to Nanosight data as shown in FIG. 26(b). Both methods show a well-aligned histogram with an average of 176±99 nm and 164±115 nm for Nanosight and plasmonic imaging based measurements.

What is claimed is:

1. A method for the detection of biological particles of interest, comprising:
fabricating one or more arrays of nanodisks, wherein the nanodisks comprise gold, a gold/silver alloy, or silver, wherein the nanodisks are about 100 nm to about 1000 nm in diameter and about 20 nm to about 150 nm in thickness, wherein the distance between nanodisks is less than the diameter of the nanodisks, and wherein the nanodisks have a blue-shifted plasmon resonance peak of at least 688 nm in air;
functionalizing the one or more arrays of nanodisks by attaching one or more recognition elements to the nanodisks, wherein the one or more recognition elements are capable of capturing the biological particles of interest, to produce one or more functionalized arrays of nanodisks;
exposing the one or more functionalized arrays of nanodisks to a sample, wherein the sample may or may not contain the biological particles of interest, to allow the recognition elements to bind biological particles of interest, wherein the biological particles of interest are biological microparticles or biological nanoparticles;
washing the one or more functionalized arrays of nanodisks to remove any unbound material from the sample, to produce one or more targeted arrays of nanodisks for visualization;
illuminating the one or more targeted arrays of nanodisks with bright field illumination to produce transmitted light;
passing the transmitted light through one or more of a condenser, an inverted microscope, an infinity corrected water immersion lens, and a bandpass filter to produce plasmonic nano-aperture transmitted light with higher or lower transmission for nanoparticle scattered light and unscattered incidence light;
using a camera device to prepare an image of the one or more targeted arrays of nanodisks, wherein the image of the one or more targeted arrays of nanodisks shows biological particles bound to the targeted array of nanodisks; and
analyzing the image of the one or more targeted arrays of nanodisks to visualize and detect biological particles of interest present in the sample.

2. The method of claim 1, wherein the nanodisks are nanoporous gold disks.

3. The method of claim 1, wherein the nanodisks in the one or more arrays of nanodisks are about 360 nm in diameter, about 50 nm in thickness, and about 100 nm apart.

4. The method of claim 1, wherein the nanodisks are positioned on undercut substrate posts.

5. The method of claim 4, wherein the undercut substrate posts are about 200 nm in diameter and about 150 nm in height.

6. The method of claim 1, wherein the recognition elements comprise proteins or oligonucleotide probes.

7. The method of claim 6, wherein the proteins comprise antibodies.

8. The method of claim 6, wherein the oligonucleotide probes comprise aptamers.

9. The method of claim 1, wherein the biological nanoparticles comprise micro-vesicles or nano-vesicles.

10. The method of claim 9, wherein the micro-vesicles or nano-vesicles comprise circulating tumor exosomes or circulating non-tumor exosomes.

11. The method of claim 1, wherein the biological nanoparticles comprise pathogens.

12. The method of claim 11, wherein the pathogens comprise bacteria or viruses.

13. The method of claim 1, further comprising a step of using spectroscopic imaging to obtain extinction spectra of the one or more targeted arrays of nanodisks.

14. The method of claim 1, further comprising a step of exposing the sample to detection elements to facilitate delivery of the detection elements into biological microparticles or biological nanoparticles in the sample prior to exposing the one or more functionalized arrays of nanodisks to the sample, wherein the detection elements hybridize to nucleic acids found in the biological particles of interest and produce fluorescence.

15. The method of claim 14, wherein the detection elements comprise molecular beacon probes.

16. The method of claim 14, further comprising a step of detecting fluorescence from the one or more targeted arrays of nanodisks after hybridization of the one or more detection elements.

17. The method of claim 16, further comprising a step of using metal-enhanced fluorescence (MEF) or surface plasmon-enhanced fluorescence (SEF) to enhance the fluorescence detected from the one or more targeted arrays of nanodisks.

18. The method of claim 1, further comprising a step of exposing the one or more targeted arrays of nanodisks to detection elements, wherein the detection elements hybridize to nucleic acids found in the biological particles of interest and produce fluorescence.

19. The method of claim 18, wherein the detection elements comprise molecular beacon probes.

20. The method of claim 18, further comprising a step of detecting fluorescence from the one or more targeted arrays of nanodisks after hybridization of the one or more detection elements.

21. The method of claim 20, further comprising a step of using metal-enhanced fluorescence (MEF) or surface plasmon-enhanced fluorescence (SEF) to enhance the fluorescence detected from the one or more targeted arrays of nanodisks.

22. The method of claim 1, further comprising a step of placing the one or more arrays of nanodisks in a microfluidic channel prior to exposing the one or more arrays of nanodisks to the sample, wherein the sample is a fluid sample comprising biological material.

23. The method of claim 22, further comprising the step of concentrating and directing the biological material in the fluid sample toward the one or more arrays of nanodisks in the microfluidic channel.

24. The method of claim 23, wherein the step of concentrating and directing the biological material is performed by nanoplasmonic microbubble and nanobubble actuators.
